(19) United States
(12) Patent Application Publication
(10) Pub. No.: US 2015/0239796 A1
(43) Pub. Date: Aug. 27, 2015

(54) ONE POT SYNTHESIS OF 18F LABELEDTRIFLUOROMETHYLATED COMPOUNDS WITH DIFLUORO(IODO)METHANE

(71) Applicant: University of Oslo, Oslo (NO)

(72) Inventors: Thomas Rühl, Belgershain (DE); Patrick Riss, Oslo (NO); Waqas Rafique, Oslo (NO)

(21) Appl. No.: 14/624,158

(22) Filed: Feb. 17, 2015

Related U.S. Application Data
(60) Provisional application No. 61/941,838, filed on Feb. 19, 2014.

(51) Int. Cl. C07B 59/00; A61K 51/04; C07C 255/50; C07D 213/26; C07D 239/54; C07C 253/30; C07D 209/12; C07D 213/74

(52) U.S. Cl. CPC C07B 59/002; C07D 209/12; A61K 51/0463; C07D 213/74; A61K 51/0459; C07D 213/26; A61K 51/0455; C07D 239/54; C07C 253/30; C07C 255/50; A61K 51/04; C07B 59/001

(57) ABSTRACT

The present invention relates to compositions and methods for the synthesis of ¹⁸F labeled compounds. In particular, the present invention relates to a copper (I) mediated one pot method for ¹⁸F-trifluoromethylation of aromatic- or heteroaromatic halides with difluoro(iodo)methane (e.g., for use at PET imaging agents).

### CROSS-REFERENCE TO RELATED APPLICATIONS

This application claims priority to pending U.S. Provisional Patent Application No. 61/941,838, filed Feb. 19, 2014, the contents of which are incorporated by reference in its entirety.

### FIELD OF INVENTION

The present invention relates to compositions and methods for the synthesis of ¹⁸F labeled compounds. In particular, the present invention relates to a copper (I) mediated one pot method for ¹⁸F-trifluoromethylation of aromatic- or heteroaromatic halides with difluoro(iodo)methane (e.g., for use at PET imaging agents).

### BACKGROUND

Numerous organic compounds bearing a trifluoromethyl group are valuable as pharmaceuticals and agrochemicals. Examples of such pharmaceuticals bearing trifluoromethyl groups include Fluoxetine (Prozac®), Celecoxib (Celebrex®), Mefloquine (Lariam®), Leflunomide (Arava®), Nilutamide (Nilandron®), Dutasteride (Avodart®), Bicalutamide (Casodex®), Aprepitant (Emend®), Flutamide (Drogenil®), Dexfenfluramine (Redux®).

Examples of such agrochemicals include Trifluralin, Fipronil, Fluazinam, Penthiopyrad, Picoxystrobin, Fluridone, and Norflurazon. Furthermore, some useful monomers, composites, materials for electronics, including electro- and photoluminescent compounds, solvents and valuable chemical building blocks and intermediates have the trifluoromethyl moiety. There is a need for simple, economic and environmentally benign methods to introduce the trifluoromethyl group into organic molecules in order to prepare active ingredients of agrochemicals and pharmaceuticals, as well as other useful compounds and materials.

Certain medical conditions, including cancer, are increasingly being diagnosed and treated using minimally invasive medical techniques. Such techniques typically involve the use of clinical imaging methods that allow the physician to visualize interior portions of a patient's body without the need to make excessive incisions. Imaging can be performed using any of variety of modalities, including, for example, X-rays, computed tomographic (CT) X-ray imaging, portal film imaging devices, electronic portal imaging devices, electrical impedance tomography (EIT), magnetic resonance (MR) imaging, or MRI, magnetic source imaging (MSI), magnetic resonance spectroscopy (MRS), magnetic resonance mammography (MRM), magnetic resonance angiography (MRA), magnetoelectro-encephalography (MEG), laser optical imaging, electric potential tomography (EPT), brain electrical activity mapping (BEAM), arterial contrast injection angiography, and digital subtraction angiography. Nuclear medicine modalities include positron emission tomography (PET) and single photon emission computed tomography (SPECT).

Some of these imaging procedures involve the use of radiographic markers. Radiographic markers are small devices that are implanted in a patient during surgical procedures, such as biopsies. Conventional markers typically consist of one or more solid objects, such as a piece of metallic wire, ceramic beads, etc., which are implanted either by themselves or within a gelatinous matrix to temporarily increase visibility, for example, to ultrasound imaging. They are designed to be visible to one of the imaging modalities listed above and typically have a shape that is readily identifiable as an artificial structure, as contrasted from naturally occurring anatomical structures in the patient's body. For example, markers can be shaped as coils, stars, rectangles, spheres, or other shapes that do not occur in anatomical structures. Such markers enable radiologists to localize the site of surgery in subsequent imaging studies or to facilitate image registration during image-guided therapeutic procedures. In this way, markers can serve as landmarks that provide a frame of reference for the radiologist.

Additional imaging agents and efficient method for generating imaging agents are needed.

### SUMMARY

The present invention relates to compositions and methods for the synthesis of ¹⁸F labeled compounds. In particular, the present invention relates to a copper (I) mediated one pot method for ¹⁸F-trifluoromethylation of aromatic- or heteroaromatic halides with difluoro(iodo)methane (e.g., for use at PET imaging agents).

For example, in some embodiments, the present invention provides a method of synthesizing a compound a compound of the formula (I) or (II) or (III) wherein Y═N, CH, or CR and wherein Z═NR, O, or S and wherein R is one or more than one and dependent or independent of each other substituted, non-substituted, functionalized, non-functionalized H, halogen, nitro, nitril, isonitril, cyanate, isocyanate, hydroxyl, amide, alkyl, alkenyl, alkynyl, aryl, heteroaryl, alkyl-, aryl-, heteroaryl ether, alkyl-, aryl-, heteroaryl thioether, alkyl-, aryl-, heteroaryl ketone, alkyl-, aryl-, heteroaryl thioketone, alkyl-, aryl-, heteroaryl amide, alkyl-, aryl-, heteroaryl thioamide, alkyl-, aryl-, heteroaryl urea, alkyl-, aryl-, heteroaryl thiourea, alkyl-, aryl-, heteroaryl urethane, alkyl-, aryl-, heteroaryl thiourethane, alkyl-, aryl-, heteroaryl ester, alkyl-, aryl-, heteroaryl thioester, alkyl-, aryl-, heteroaryl amine, monocyclic, or multi cyclic, isotope containing and wherein n=1-4,

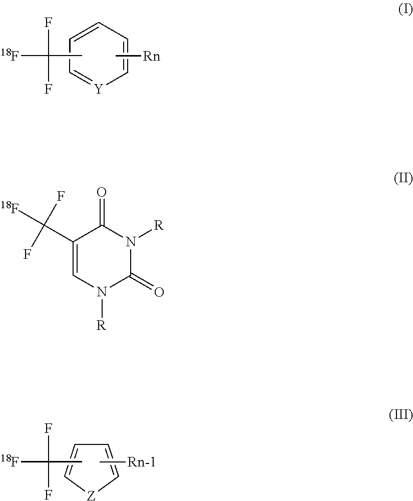

in a single reaction vessel containing a base, ¹⁸F⁻ ion and a copper source, contacted with difluoro(iodo)methane and a compound of the formula (IV) or (V) or (VI) wherein Y═N, CH, CR and wherein Z═NR, O, or S and wherein X═Cl, Br, or I and wherein R is one or more than one and dependent or independent of each other substituted, non-substituted, functionalized, non-functionalized H, halogen, nitro, nitril, isonitril, cyanate, isocyanate, hydroxyl, amide, alkyl, alkenyl, alkynyl, aryl, heteroaryl, alkyl-, aryl-, heteroaryl ether, alkyl-, aryl-, heteroaryl thioether, alkyl-, aryl-, heteroaryl ketone, alkyl-, aryl-, heteroaryl thioketone, alkyl-, aryl-, heteroaryl amide, alkyl-, aryl-, heteroaryl thioamide, alkyl-, aryl-, heteroaryl urea, alkyl-, aryl-, heteroaryl thiourea, alkyl-, aryl-, heteroaryl urethane, alkyl-, aryl-, heteroaryl thiourethane, alkyl-, aryl-, heteroaryl ester, alkyl-, aryl-, heteroaryl thioester, alkyl-, aryl-, heteroaryl amine, monocyclic, or multi cyclic, isotope containing and wherein n=1-4,

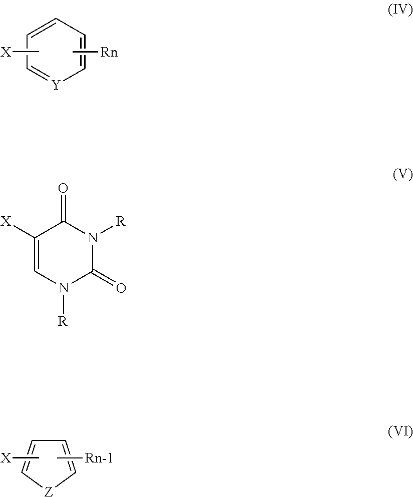

and a ligand, in a solvent in a solvent for an appropriate incubation time at an elevated temperature. In some embodiments, the temperature is between 50° C. and 750° C. (e.g., 145° C.). In some embodiments, the incubation time is between 1 s and 5 h (e.g., 10 minutes). In some embodiments, the copper source is a copper(I) source (e.g., CuBr, Tetrakisacetonitrile copper(I) triflate or CuI). In some embodiments, the solvent is a polar aprotic solvent (e.g., DMF, acetonitrile, or dialkyl ketone). In some embodiments, the base is a metal carbonate and/or metal bicarbonate and cyptand (e.g., KHCO₃ and crypt-222, Cs₂CO₃ and crypt-222, K₂CO₃ and crypt-222, K₂CO₃ and 18-Crown-6, a nonmetal carbonate, a nonmetal bicarbonate, or tetraethylammonium bicarbonate). In some embodiments, the ligand is an organic non- to low-nucleophilic amine or phosphazene (e.g., DBU, TMEDA, NEt₃ or DIPEA). In some embodiments, the ligand stabilized the copper mediate. In some embodiments, the ligand is a base. In some embodiments, when ¹⁹F-fluoride ion is present, the CF₃ substituted compounds are also synthesized.

Further embodiments provide compounds synthesized by the methods described herein, and methods of using the compounds as PET imaging agents.

Additional embodiments are described herein.

### BRIEF DESCRIPTION OF THE DRAWINGS

These and other features, aspects, and advantages of the present technology will become better understood with regard to the following drawings:

FIG. 1 shows NMR spectra with 4-trifluoromethylbenzonitrile at around 7.72 ppm and ether at 7.55 and 7.02.

FIG. 2 shows NMR of a sample containing CuCl, t-BuOK and CHF3 in DMF. The signal around −26 is CuCF3 and the signal around −81 is CHF3.

FIG. 3 shows an exemplary setup for parallel evaporations.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, the terms “subject” and “patient” refer to any animal, such as a mammal like a dog, cat, bird, livestock, and preferably a human.

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the term “administration” refers to the act of giving a drug, prodrug, or other agent, or therapeutic treatment to a subject. Exemplary routes of administration to the human body can be through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.), and the like.

As used herein, the term “co-administration” refers to the administration of at least two agents or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present technology.

As used herein, the terms “alkyl” and the prefix “alk-” are inclusive of both straight chain and branched chain saturated or unsaturated groups, and of cyclic groups, e.g., cycloalkyl and cycloalkenyl groups. Unless otherwise specified, acyclic alkyl groups are from 1 to 6 carbons. Cyclic groups can be monocyclic or polycyclic and preferably have from 3 to 8 ring carbon atoms. Exemplary cyclic groups include cyclopropyl, cyclopentyl, cyclohexyl, and adamantyl groups. Alkyl groups may be substituted with one or more substituents or unsubstituted. Exemplary substituents include alkoxy, aryloxy, sulfhydryl, alkylthio, arylthio, halogen, alkylsilyl, hydroxyl, fluoroalkyl, perfluoralkyl, amino, aminoalkyl, disubstituted amino, quaternary amino, hydroxyalkyl, carboxyalkyl, and carboxyl groups. When the prefix “alk” is used, the number of carbons contained in the alkyl chain is given by the range that directly precedes this term, with the number of carbons contained in the remainder of the group that includes this prefix defined elsewhere herein. For example, the term “C₁-C₄ alkaryl” exemplifies an aryl group of from 6 to 18 carbons (e.g., see below) attached to an alkyl group of from 1 to 4 carbons.

As used herein, the term “aryl” refers to a carbocyclic aromatic ring or ring system. Unless otherwise specified, aryl groups are from 6 to 18 carbons. Examples of aryl groups include phenyl, naphthyl, biphenyl, fluorenyl, and indenyl groups.

As used herein, the term “heteroaryl” refers to an aromatic ring or ring system that contains at least one ring heteroatom (e.g., O, S, Se, N, or P). Unless otherwise specified, heteroaryl groups are from 1 to 9 carbons. Heteroaryl groups include furanyl, thienyl, pyrrolyl, imidazolyl, pyrazolyl, oxazolyl, isoxazolyl, thiazolyl, isothiazolyl, triazolyl, tetrazolyl, oxadiazolyl, oxatriazolyl, pyridyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl groups.

As used herein, the term “heterocycle” refers to a non-aromatic ring or ring system that contains at least one ring heteroatom (e.g., O, S, Se, N, or P). Unless otherwise specified, heterocyclic groups are from 2 to 9 carbons. Heterocyclic groups include, for example, dihydropyrrolyl, tetrahydropyrrolyl, piperazinyl, pyranyl, dihydropyranyl, tetrahydropyranyl, dihydrofuranyl, tetrahydrofuranyl, dihydrothiophene, tetrahydrothiophene, and morpholinyl groups.

Aryl, heteroaryl, or heterocyclic groups may be unsubstituted or substituted by one or more substituents selected from the group consisting of C₁₋₆ alkyl, hydroxy, halo, nitro, C₁₋₆ alkoxy, C₁₋₆ alkylthio, trifluoromethyl, C₁₋₆ acyl, arylcarbonyl, heteroarylcarbonyl, nitrile, C₁₋₆ alkoxycarbonyl, alkaryl (where the alkyl group has from 1 to 4 carbon atoms), and alkheteroaryl (where the alkyl group has from 1 to 4 carbon atoms).

As used herein, the term “alkoxy” refers to a chemical substituent of the formula —OR, where R is an alkyl group. By “aryloxy” is meant a chemical substituent of the formula —OR′, where R′ is an aryl group.

As used herein, the term “C_{x-y} alkaryl” refers to a chemical substituent of formula RR′, where R is an alkyl group of x to y carbons and R′ is an aryl group as defined elsewhere herein.

As used herein, the term “C_{x-y} alkheteraryl” refers to a chemical substituent of formula RR″, where R is an alkyl group of x to y carbons and R″ is a heteroaryl group as defined elsewhere herein.

As used herein, the term “halide” or “halogen” or “halo” refers to bromine, chlorine, iodine, or fluorine.

As used herein, the term “non-vicinal O, S, or N” refers to an oxygen, sulfur, or nitrogen heteroatom substituent in a linkage, where the heteroatom substituent does not form a bond to a saturated carbon that is bonded to another heteroatom.

For structural representations where the chirality of a carbon has been left unspecified it is to be presumed by one skilled in the art that either chiral form of that stereo center is possible.

### EMBODIMENTS OF THE TECHNOLOGY

The present invention relates to compositions and methods for the synthesis of ¹⁸F labeled compounds. In particular, the present invention relates to a copper (I) mediated one pot method for ¹⁸F-trifluoromethylation of aromatic- or heteroaromatic halides with difluoro(iodo)methane (e.g., for use at PET imaging agents).

Molecular imaging with positron emission tomography (PET) allows for non-invasive, quantitative studies of radiotracer distribution in living subjects. In consequence of its maturation, PET is increasingly used in routine clinical diagnosis, commercial drug development, and in biomedical research. Novel radiotracers for imaging a variety of biological targets are continually needed to fully exploit the potential of PET. ¹⁸F is the most frequently employed PET nuclide, owed to the extensive use of 2-[¹⁸F]fluoro-2-deoxy-D-glucose ([¹⁸F]FDG) for clinical diagnosis.2,3 The relevance of ¹⁸F is based on its expedient half life (109.7 min) rendering it suitable for multi-step reactions, transport of radiotracers over moderate distances, convenient handling of the tracer in imaging studies and high-yield cyclotron production of no carrier added (n.c.a) [¹⁸F]fluoride ion. The ability to form stable C—F bonds promotes the straight introduction of F atoms into most small organic molecules. Despite the strong demand for novel radiotracers for a variety of disease related biological processes, radiotracer development is impeded by a complex process. Researchers and clinicians often struggle to obtain a desired radiotracer within a reasonable time frame because discovery of suitable molecular structures that can be labelled by established procedures often require time-consuming iterative cycles of candidate synthesis and biological evaluation.

Solutions to overcome this bottleneck include: a) Development of more versatile labelling processes with the most appropriate radionuclides to increase the number of molecular structures that can be labelled; and b) Sourcing radiotracer candidates from known, well characterized drug molecules in combination with new labelling methods to avoid iterative optimization of molecular scaffolds and streamline the work-flow. Efficient methodology for nucleophilic radiofluorination of the trifluoromethyl group is seen to be key to both of these solutions. A wide portfolio of known drug molecules contain the metabolically stable CF3 group, most of which may be susceptible for repurposing as PET radiotracers (D. A. Nagib, D. W. C. MacMillan. Nature 2011, 480, 224-228; A. Deb et al., Chem Int Ed 2013, 52: 9747-9750; S. Mizuta et al., Nature 211, 473, 470-477; S. Mizuta et al., J. Am. Chem. Soc. 2013, 135 (7), 2505-2508; M. Huiban et al., Nature Chem. 2013 5, 941-944; L. Zhu et al., Org Lett. 2013, 15 (11), 2648-2651; M. Tredwell et al., Chem. Int. Ed. 2012, 51(46), 11426-11437; P. J. Riss et al., Org. Biomol. Chem., 2012, 10, 6980-6986; P. J. Riss et al., 2011, 47, 11873-11875; M. R. Kilbourn et al., Int. J. Rad. Appl. Instrum. A, 1990, 41, 823-828; O. Josse et al., Bioorg. Med. Chem. 2001, 9, 665-675; W. R. Dolbier Jr et al., Appl. Radiat. Isotopes 2001, 54:73-80).

CF₃ groups are found in abundance in drug molecules and operationally simple, direct arene-trifluoromethylation methodology has become a key focus in current organic chemistry (e.g. MacMillan in Nature 2011) (D. A. Nagib, D. W. C. MacMillan. Nature 2011, 480, 224-228; A. Deb et al., Chem Int Ed 2013, 52: 9747-9750; S. Mizuta et al., Nature 211, 473, 470-477; S. Mizuta et al., J. Am. Chem. Soc. 2013, 135 (7), 2505-2508). The C—F bond in CF₃ groups is attractive for radiolabelling to access known drug molecules for PET and for introduction of a metabolically insensitive radiolabel (M. Huiban et al., Nature Chem. 2013 5, 941-944; L. Zhu et al., Org Lett. 2013, 15 (11), 2648-2651; M. Tredwell et al., Chem. Int. Ed. 2012, 51(46), 11426-11437; P. J. Riss et al., Org. Biomol. Chem., 2012, 10, 6980-6986; P. J. Riss et al., 2011, 47, 11873-11875; M. R. Kilbourn et al., Int. J. Rad. Appl. Instrum. A, 1990, 41, 823-828; O. Josse et al., Bioorg. Med. Chem. 2001, 9, 665-675; W. R. Dolbier Jr et al., Appl. Radiat. Isotopes 2001, 54:73-80). An efficient method for producing [¹⁸F]trifluoromethyl arenes starting from [¹⁸F]fluoride ion was developed. Radiosynthesis of the ¹⁸F-labelled aryl trifluoromethane scaffold has been reported, however, mostly through the use of rare and inavailable electrophilic fluorinating agents or harsh conditions (M. Huiban et al., Nature Chem. 2013 5, 941-944; L. Zhu et al., Org Lett. 2013, 15 (11), 2648-2651; M. Tredwell et al., Chem. Int. Ed. 2012, 51(46), 11426-11437; P. J. Riss et al., Org. Biomol. Chem., 2012, 10, 6980-6986; P. J. Riss et al., 2011, 47, 11873-11875; M. R. Kilbourn et al., Int. J. Rad. Appl. Instrum. A, 1990, 41, 823-828; O. Josse et al., Bioorg. Med. Chem. 2001, 9, 665-675; W. R. Dolbier Jr et al., Appl. Radiat. Isotopes 2001, 54:73-80). A more recent breakthrough employed CuI in combination with aryl iodides (Huiban et al., supra). Described herein is a new route utilizing [¹⁸F]fluoroform as an intermediate by Vugts et al. (Chem. Commun., 2013, 49, 4018-4020).

For successful outcomes, reactions involving [¹⁸F]fluoroform utilize diligent control of the gaseous intermediate, including low temperature distillation and trapping of the product at −60-100° C. in a secondary reaction vessel. These conditions and technical requirements are limiting factors with respect to the automated synthesis of high activity batches using automated synthesiser systems. Few commercially available systems provide more than one reactor and generally disfavour low temperature processes. It was determined that widespread adaption of trifluoromethylation reactions would strongly benefit from a straightforward nucleophilic one-pot method generally applicable to latest generation synthetic hardware. Such methodology would furthermore feature direct installation of nucleophilic fluorine-18 in the form of n.c.a. [¹⁸F]fluoride ion into candidate radiotracers to avoid losses of radioactivity, conserve specific radioactivity and achieve rapid and simple radiosyntheses.

A copper(I) mediated [18F]-trifluoromethylation with [18F]-fluoroform at 100° C. (D. A. Nagib, D. W. C. MacMillan. Nature 2011, 480, 224-228; A. Deb et al., Chem Int Ed 2013, 52: 9747-9750; S. Mizuta et al., Nature 211, 473, 470-477; S. Mizuta et al., J. Am. Chem. Soc. 2013, 135 (7), 2505-2508) was investigated. [18F]-fluoroform was prepared from [18F]-fluoride and difluoro(iodo)methane at room temperature (L. Cai, S. Lu, V. W. Pike, Eur. J. Org. Chem. 2008, 2853-2873). A disadvantage of this method is that [18F]-fluoroform has a boiling point of −84° C., e.g. it is a radioactive gas at room temperature, which complicates the procedure. Difluoro(iodo)methane has a boiling point of 22° C.

The expectations were that the difluoro(iodo)methane and the [18F]-fluoroform in DMF would outgas at an elevated temperature. Contrary to the expectations of outgassing, the copper(I) mediated preparation of the ¹⁸F-trifluoromethylated products from [18F]-fluoride and difluoro(iodo)methane in a one pot reaction worked excellent in the presence of non- to low-nucleophilic amines. In the context of the nuclide fluorine-18 short half-life of only 110 min, this is an enormous time-saver.

The published ¹⁸F-trifluoromethylation with [18F]-fluoroform needs a strong base like tert-BuOK for the deprotonation of the fluoroform and to form the copper intermediate. In the case of the one pot reaction, it was found that strong bases impeded the reaction. Only traces of ¹⁸F-trifluoromethylatet product were observed. The base which gave the best results was N,N-diisopropylethylamine (DIPEA). DIPEA and also the carbonates which was used for the one pot [18F]-trifluoromethylation method are not strong enough for deprotonating of [18F]-fluoroform. This indicates that the reaction does involve [18F]-fluoroform as an intermediate, because it can be formed but after that not deprotonated under the conditions. Completely unexpectedly, it was that there was no impairment by copper mediated ¹⁸F-fluorination of aryl iodides with difluoro(iodo)methane.

The formation of difluorocarbene with methyl chlorodifluoroacetate and CuI is published by Su et al. (ournal of the Chemical Society, Chem. Commun. 1992, 11, 807-808) and McNail et al. (J. Fluorine Chem. 1991, 55, 225). The formation of fluoroform with methyl chlorodifluoroacetate is unknown.

It was found that the method involves the cleavage of the carbon-hydrogen bond and of the iodine substituent of difluoro(iodo)methane. The use of methyl chlorodifluoroacetate involves a iodide mediated Krapcho demethylation as initial reaction step followed by decarboxylative formation of difluorocarbene. This is a sequence of two different reactions which are not possible with difluoro(iodo)methane. Hence, it was not foreseeable that the one pot ¹⁸F-trifluoromethylation method would work with difluoro(iodo)methane. Methyl chlorodifluoroacetate has a boiling point of 79-81° C. The loss of chlorodifluoroacetate through outgassing from a DMF solution at 150° C. was not probable.

Accordingly, in some embodiments, the present invention provides a copper(I) mediated one pot method for [18F]-trifluoromethylation of aromatic- or heteroaromatic halides with difluoro(iodo)methane and ¹⁸F-ion.

In some embodiments, the present invention provides a method of synthesizing a compound of the formula (I) or (II) or (III) wherein Y═N, CH, CR and wherein Z═NR, O, S and wherein R is one or more than one and dependent or independent of each other substituted, non-substituted, functionalized, non-functionalized H, halogen, nitro, nitril, isonitril, cyanate, isocyanate, hydroxyl, amide, alkyl, alkenyl, alkynyl, aryl, heteroaryl, alkyl-, aryl-, heteroaryl ether, alkyl-, aryl-, heteroaryl thioether, alkyl-, aryl-, heteroaryl ketone, alkyl-, aryl-, heteroaryl thioketone, alkyl-, aryl-, heteroaryl amide, alkyl-, aryl-, heteroaryl thioamide, alkyl-, aryl-, heteroaryl urea, alkyl-, aryl-, heteroaryl thiourea, alkyl-, aryl-, heteroaryl urethane, alkyl-, aryl-, heteroaryl thiourethane, alkyl-, aryl-, heteroaryl ester, alkyl-, aryl-, heteroaryl thioester, alkyl-, aryl-, heteroaryl amine, monocyclic, multi cyclic, isotope containing and wherein n=1-4,

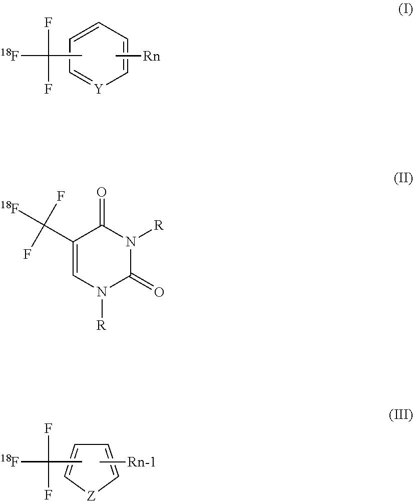

in a single reaction vessel containing a base, ¹⁸F⁻ ion and a copper source, contacted with difluoro(iodo)methane and a compound of the formula (IV) or (V) or (VI) wherein Y═N, CH, CR and wherein Z═NR, O, S and wherein X═Cl, Br, I and wherein R is one or more than one and dependent or independent of each other substituted, non-substituted, functionalized, non-functionalized H, halogen, nitro, nitril, isonitril, cyanate, isocyanate, hydroxyl, amide, alkyl, alkenyl, alkynyl, aryl, heteroaryl, alkyl-, aryl-, heteroaryl ether, alkyl-, aryl-, heteroaryl thioether, alkyl-, aryl-, heteroaryl ketone, alkyl-, aryl-, heteroaryl thioketone, alkyl-, aryl-, heteroaryl amide, alkyl-, aryl-, heteroaryl thioamide, alkyl-, aryl-, heteroaryl urea, alkyl-, aryl-, heteroaryl thiourea, alkyl-, aryl-, heteroaryl urethane, alkyl-, aryl-, heteroaryl thiourethane, alkyl-, aryl-, heteroaryl ester, alkyl-, aryl-, heteroaryl thioester, alkyl-, aryl-, heteroaryl amine, monocyclic, multi cyclic, isotope containing and wherein n=1-4,

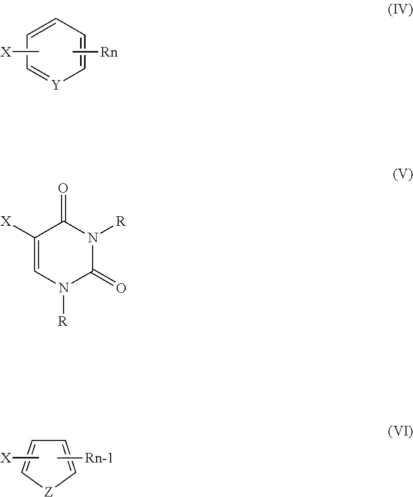

and a ligand, in a solvent in a solvent for an appropriate incubation time at an elevated temperature. In some embodiments, the temperature is 145° C. In some embodiments, the incubation time is approximately 10 minutes. The present invention is not limited to particular copper sources, solvents, bases, and ligands. Exemplary options are described herein. For example, in some embodiments, the copper source is CuBr, the solvent is DMF, the base is KHCO₃ and crypt-222, and the ligand DBU, TMEDA, NEt₃ or DIPEA. The present invention is not limited to a particular R groups (e.g., those shown in Table 5).

A general procedure is:

[¹⁸F]fluoride ion (1 ml dilution in water) was trapped on a Sep-Pak® Accell® Plus QMA Plus Light Cartridge which was before additional conditioned with 1M potassium carbonate solution (5 ml), rinsed with water (10 ml) and rinsed with air (30 ml). After rinsing with air (10 ml), the [¹⁸F]fluoride ion was eluted to a vessel using a solution of Kryptofix K2.2.2 (35 μM) and KHCO3 (13 μM) in MeCN/H2O=9/1 (1 mL) following by portioning. The solutions were evaporated to dryness in a stream of Argon at 90° C. for 5 min. Residual water was removed by azeotropic co-evaporation using two portions of anhydrous MeCN (2×1 mL) for 5 min each.

CuBr (58 μmol) was added to [¹⁸F]KF and the vial was flushed with argon for 2 min. An ice cooled solution of DIPEA (59 μmol), aryliodide (38 μmol) and difluoro(iodo)methane (169 μmol) in dry DMF (300 μl) were added via syringe. The sealed vial was heated at 145° C. for 10 min.

The imaging agents of the present technology find many uses. In particular, the imaging agents of the present technology find use as imaging agents within nuclear medicine imaging protocols (e.g., PET imaging, SPECT imaging).

In preferred embodiments, radiotracers of the present technology are useful as imaging agents within PET imaging studies. PET is the study and visualization of human physiology by electronic detection of short-lived positron emitting radiopharmaceuticals. It is a non-invasive technology that quantitatively measures metabolic, biochemical, and functional activity in living tissue.

The PET scan is a vital method of measuring body function and guiding disease treatment. It assesses changes in the function, circulation, and metabolism of body organs. Unlike MRI (Magnetic Resonance Imaging) or CT (Computed Tomography) scans that primarily provide images of organ anatomy, PET measures chemical changes that occur before visible signs of disease are present on CT and MRI images.

PET visualizes behaviors of trace substances within a subject (e.g., a living body) having a radioimaging agent administered therein by detecting a pair of photons occurring as an electron/positron annihilation pair and moving in directions opposite from each other (see, e.g., U.S. Pat. No. 6,674,083, herein incorporated by reference in its entirety). A PET apparatus is equipped with a detecting unit having a number of small-size photon detectors arranged about a measurement space in which the subject is placed. The detecting unit detects frequencies of the generation of photon pairs in the measurement space on the basis of the stored number of coincidence-counting information items, or projection data, and then stores photon pairs occurring as electron/positron annihilation pairs by coincidence counting and reconstructs an image indicative of spatial distributions. The PET apparatus plays an important role in the field of nuclear medicine and the like, whereby biological functions and higher-order functions of brains can be studied by using it. Such PET apparatuses can be roughly classified into two-dimensional PET apparatuses, three-dimensional PET apparatuses, and slice-septa-retractable type three-dimensional PET apparatuses.

In general, a PET detector or camera typically consists of a polygonal or circular ring of radiation detectors placed around a patient area (see, e.g., U.S. Pat. No. 6,822,240, herein incorporated by reference in its entirety). Radiation detection begins by injecting isotopes with short half-lives into a patient's body placed within the patient area. The isotopes are absorbed by target areas within the body and emit positrons. In the human body, the positrons annihilate with electrons. As a result thereof, two essentially monoenergetic gamma rays are emitted simultaneously in opposite directions. In most cases the emitted gamma rays leave the body and strike the ring of radiation detectors.

The ring of detectors includes typically an inner ring of scintillation crystals and an outer ring of light detectors, e.g., photomultiplier tubes. The scintillation crystals respond to the incidence of gamma rays by emitting a flash of light (photon energy), so-called scintillation light, which is then converted into electronic signals by a corresponding adjacent photomultiplier tube. A computer, or similar, records the location of each light flash and then plots the source of radiation within the patient's body by comparing flashes and looking for pairs of flashes that arise simultaneously and from the same positron-electron annihilation point. The recorded data is subsequently translated into a PET image. A PET monitor displays the concentration of isotopes in various colors indicating level of activity. The resulting PET image then indicates a view of neoplasms or tumors existing in the patient's body.

Such detector arrangement is known to have a good energy resolution, but relatively bad spatial and temporal resolutions. Early PET detectors required a single photomultiplier tube to be coupled to each single scintillation crystal, while today, PET detectors allow a single photodetector to serve several crystals, see e.g. U.S. Pat. Nos. 4,864,138; 5,451,789; and 5,453,623, each herein incorporated by reference in their entireties). In such manner the spatial resolution is improved or the number of photodetectors needed may be reduced.

Single Photon Emission Computed Tomography (SPECT) is a tomographic nuclear imaging technique producing cross-sectional images from gamma ray emitting radiopharmaceuticals (single photon emitters or positron emitters). SPECT data are acquired according to the original concept used in tomographic imaging: multiple views of the body part to be imaged are acquired by rotating the Anger camera detector head(s) around a craniocaudal axis. Using backprojection, cross-sectional images are then computed with the axial field of view (FOV) determined by the axial field of view of the gamma camera. SPECT cameras are either standard gamma cameras that can rotate around the patient's axis or consist of two or even three camera heads to shorten acquisition time. Data acquisition is over at least half a circle (180°) (used by some for heart imaging), but usually over a full circle. Data reconstruction takes into account the fact that the emitted rays are also attenuated within the patient, e.g., photons emanating from deep inside the patient are considerably attenuated by surrounding tissues. While in CT absorption is the essence of the imaging process, in SPECT attenuation degrades the images. Thus, data of the head reconstructed without attenuation correction may show substantial artificial enhancement of the peripheral brain structures relative to the deep ones. The simplest way to deal with this problem is to filter the data before reconstruction. A more elegant but elaborate method used in triple head cameras is to introduce a gamma-ray line source between two camera heads, which are detected by the opposing camera head after being partly absorbed by the patient. This camera head then yields transmission data while the other two collect emission data. Note that the camera collecting transmission data has to be fitted with a converging collimator to admit the appropriate gamma rays.

SPECT is routinely used in clinical studies. For example, SPECT is usually performed with a gamma camera comprising a collimator fixed on a gamma detector that traces a revolution orbit around the patient's body. The gamma rays, emitted by a radioactive tracer accumulated in certain tissues or organs of the patient's body, are sorted by the collimator and recorded by the gamma detector under various angles around the body. From the acquired planar images, the distribution of the activity inside the patient's body is computed using certain reconstruction algorithms. Generally, the so-called Expectation-Maximization of the Maximum-Likelihood (EM-ML) algorithm is used, as described by Shepp et al. (IEEE Trans. Med. Imaging 1982; 2:113-122) and by Lange et al. (J. Comput. Assist. Tomogr. 1984; 8:306-316). This iterative algorithm minimizes the effect of noise in SPECT images.

In some embodiments, the imaging agents of the present technology are used as imaging agents for PET imaging.

It is contemplated that the imaging agents of the present technology are provided to a nuclear pharmacist or a clinician in kit form.

A pharmaceutical composition produced according to the present technology comprises use of one of the aforementioned imaging agents and a vehicle such as a physiological buffered saline solution a physiologically buffered sodium acetate carrier. It is contemplated that the composition will be systemically administered to the patient as by intravenous injection. For example in the case of fludeoxyglucose-18F, the figures for systems with bed overlap of <25% are:

Product of MBq/kg×min/bed>27.5 for 2D scans

Product of MBq/kg×min/bed>13.8 for 3D scans.

The dosage is then calculated as follows:

FDG activity in MBq for 2D scans=27.5×weight/(min/bed)

FDG activity in MBq for 3D scans=13.8×weight/(min/bed)

And for systems with a bed overlap of 50%:

Product of MBq/kg×min/bed>6.9 (3D only)

FDG activity in MBq=6.9×weight/(min/bed)¹³

Suitable dosages for use as a diagnostic imaging agent are, for example, from about 2 mCi (74 MBq) to about 20 mCi (740 MBq) of F-18 labeled imaging agent for the whole body or peripheral organs, and from about 2.0 to about 20.0 mCi of the F-18 labeled agent for imaging of the brain.

It will be appreciated by those skilled in the art that the imaging agents of the present technology are employed in accordance with conventional methodology in nuclear medicine in a manner analogous to that of the aforementioned imaging agents. Thus, a composition of the present technology is typically systemically applied to the patient, and subsequently the uptake of the composition in the selected organ is measured and an image formed, for example, by means of a conventional gamma camera.

Although the disclosure herein refers to certain illustrated embodiments, it is to be understood that these embodiments are presented by way of example and not by way of limitation.

### EXAMPLES

### Experimental Section

Typical Reaction.

[¹⁸F]fluoride ion in water was produced by the 18O(p,n)¹⁸F nuclear reaction using a GE PETtrace cyclotron. After irradiation, [¹⁸F]fluoride ion (5.1 GBq in 1 ml of water) was trapped on a Sep-Pak® Accell® Plus QMA Plus Light Cartridge. After purging with air (10 ml), the [¹⁸F]fluoride ion (4.5 GBq) was eluted into a reactor using a solution of Kryptofix K2.2.2 (35 μM) and KHCO3 (13 μM) in MeCN—H2O, 9:1 (1 mL). The solutions were evaporated to dryness in a stream of Argon at 90° C. for 5 min. Residual water was removed by azeotropic co-evaporation using two portions of anhydrous MeCN (2×1 mL) for 5 min per portion. CuBr (8 mg, 58 μmol) was added to the residue and the reaction vessel was flushed with argon for 2 min. An ice cooled solution of DIPEA (10 μl, 59 μmol), [6-iodo-2-methyl-1-(2-morpholinoethyl) indol-3-yl]-(4-methoxyphenyl)methanone (11 mg, 21 μmol) and difluoro(iodo)methane (20 μl in 169 μmol) in dry DMF (300 μl) was added and the sealed reaction vessel was heated to 145° C. for 10 min. The reaction was terminated and the cooled reaction mixtures was injected into radio HPLC and analyzed by radio TLC (n-hexane-ethyl acetate, 1:1).

[¹⁸F]-(4-methoxyphenyl)-[2-methyl-1-(2-morpholinoethyl)-6-(trifluoromethyl)indol-3-yl]methanone was isolated via solid phase extraction on a Waters SepPak C18 cartridge in a yield of up to 42%.

Materials.

Aromatic- and heteroaromatic halides were purchased from Sigma-Aldrich (Sigma-Aldrich Norway AS, Oslo, Norway) or VWR International (VWR International AS, Oslo, Norge). Copper salts were obtained from Sigma-Aldrich in either high purity (99.999%) or ‘purum’ quality (99.5%). All other chemicals and solvents were purchased in highest available purity and used as received unless stated otherwise. Difluoro(iodo)methane was obtained from Fluorochem (Fluorochem Ltd., Hadfield, Derbyshire, UK) and purified by distillation after drying over molecular sieve A4.

General.

All purchased chemicals were used as specified below Anhydrous solvents were used for reactions involving [¹⁸F]fluoride ion. ¹H— (400 MHz), 13C— (100 MH) and ¹⁹F— (376 MHz) NMR spectra were recorded on an Bruker AVII 400 NMR spectrometer (Bruker AXS Nordic AB, Solna, Sweden). Chemical shifts (δ) for proton and carbon resonances are reported in parts per million (ppm) downfield from tetramethylsilane (TMS, δ=0 ppm). ¹⁹F-NMR spectra were referenced to external CFCl₃ (δ=0 ppm) in the indicated solvent. Analytical HPLC was performed on an apparatus (Wissenschaftliche Gerätebau Dr. Ing. Herbert Knauer GmbH, Berlin, Germany) comprised of a binary pump and a variable wavelength UV detector using Chromstar software for data acquisition and analysis. The system was equipped with a Chromolith RP18e column (5 μm; 100 Å; 100 mm×4.6 mm; VWR, Darmstadt, Germany) or a Supelco FS-5 column (5 μm; 100 Å; 250 mm×4.6 mm, Sigma-Aldrich Norway AS, Oslo, Norway). UV absorption was detected at 254 nm. Three isocratic mobile phases were used: System A: MeCN—H₂O, 3:7; System B: MeCN—H₂O, 1:1; System C: MeCN—H₂O, 7:3 at a volume flow rate of 3.0 mL/min for screening reactions. Isocratic conditions (Method B: MeCN—H₂O, 30:70 v/v; 3 mL/min; was used for the analyses of [18F](4-methoxyphenyl)-[2-methyl-1-(2-morpholinoethyl)-6-(trifluoromethyl)indol-3-yl]methanone, [18F]-1-[5-(trifluoromethyl)-2-pyridyl]piperazine, [18F]1,3-dimethyl-5-(trifluoromethyl)pyrimidine-2,4-dione. Radioactivity was measured for quantification of radiochemical yields with an Atomlab 300 dose calibrator (Biodex Medical Systems). RadioTLC was conducted on Kieselgel 60 HF254 TLC plates (Merck, Darmstadt, Germany). Detection was performed using a raytest mini Gita radioTLC scanner and raytest Gina Star software (Raytest GmbH, Straubenhardt, Germany).

Syntheses and Non-Radioactive Control Experiments

Reactions with Fluoroform, CHF₃

Initial experiments were conducted using commercially available fluoroform under stoichiometric conditions. All manipulations were made without using a glovebox, and all reagents were used as purchased without further purification. Various conditions for trifluoromethylation based on the published procedure by Grushin et al. (WO 2012/113726) (scheme 1) were tested.

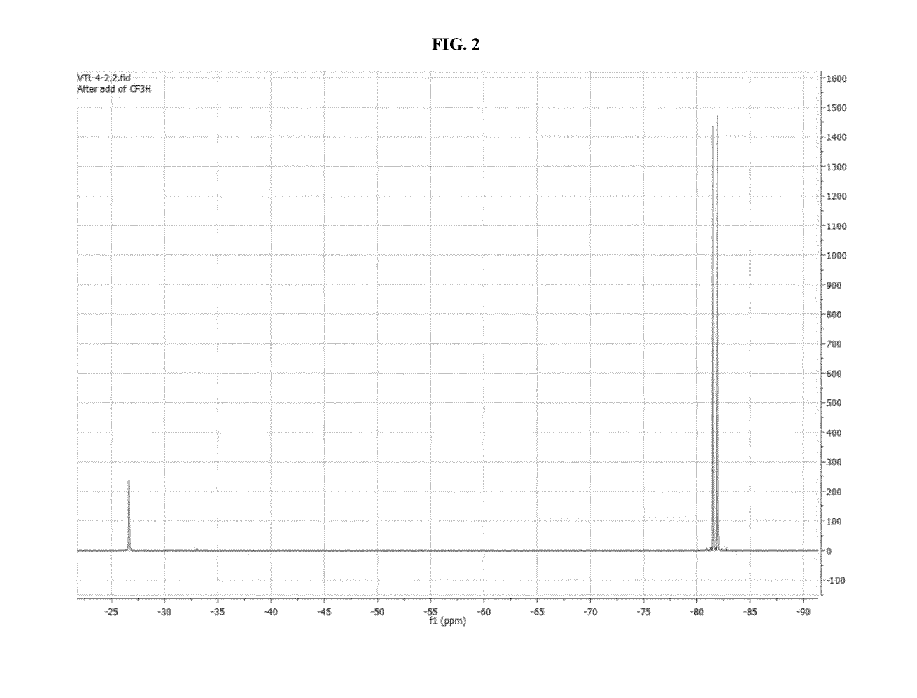

These authors initiated their investigations using phenanthroline as a ligand but later reported a ligand-free system wherein 2 equivalents of t-BuOK relative to the catalyst are sufficient to achieve high yields. However, successful outcomes require super-stoichiometric amounts of triethylamine trihydrofluoride (TREAT-HF) to be added to stabilize the intermediate Cu—CF3 species. TREAT-HF is also reported to supress byproduct formation through unwanted C—O bond formation to furnish tert-butyl-aryl ethers. Without additives this competing side product is reported in an 1:1.2 ratio with respect to the desired product (product:ether). This is problematic in the context of no carrier added radiochemistry, because superstoichiometric amounts of fluorine confound the specific activity of the final product. Hence the initial effort was focused on eliminating the need for additives to render the reaction more useful for PET chemistry. Indeed, this unwanted side reaction in absence of TREAT-HF was confirmed, as shown in the NMR spectra in FIG. 2 (with 4-trifluoromethylbenzonitrile). In order to avoid ether formation, stoichiometry of t-BuOK and ligand was investigated. This gave a slight reduction of the formed ether (1:1), but the yields of the trifluoromethylated products were also markedly reduced from around 40% to 15%. See Table 1: entries 1 and 2. Neither replacement of one equivalent of t-BuOK by pyridine, nor phenanthroline had a beneficial effect on product distribution.

TABLE 1
Copper mediated trifluoromethylation

| Entry | R | Eq. t-BuOK | Eq. Phen | Yield (%) | prod:ether |
|---|---|---|---|---|---|
| 1 | 4-CN | 2 | 0 | 40 | 1:4 |
| 2 | 4-CN | 1 | 1 | 16 | 1:1 |
| 3 | 4-Ph | 1 | 1 | 16 | Unknown |
| 4 | 4-H₂NCO | 1 | 1 | 10 | Unknown |
| 5 | 4-CN | 3 (K₂CO₃) | 1 | 0 | — |
| 6 | 4-CN | 2 (TMEDA) | 0 | 0 | — |

Notably, the transfer of gaseous fluoroform into the reaction mixture was found to be a critical step and a large excess had to be employed, presumably due to poor solubility of the reagent in DMF at room temperature. Yields comparable to Grushin et al. could not be achieved with an easy bubbling procedure, which was also confirmed doing hot chemistry. When tested with ¹⁸F-labelled fluoroform as a tracer, considerable loss of activity was observed during the trapping process. Even at low temperature (e.g. −80° C.) and careful addition at low flow rate (15 mL/min), trapping efficiency was far from optimal and up to 20% of the fluoroform were found in gas traps behind the trapping vessel. Following the initial results with K₂CO₃ and TMEDA in radiochemical experiments, these reagents were also tested with commercial fluoroform, albeit without success (entries 5 and 6).

General Procedure for Reactions Involving Fluoroform:1,2

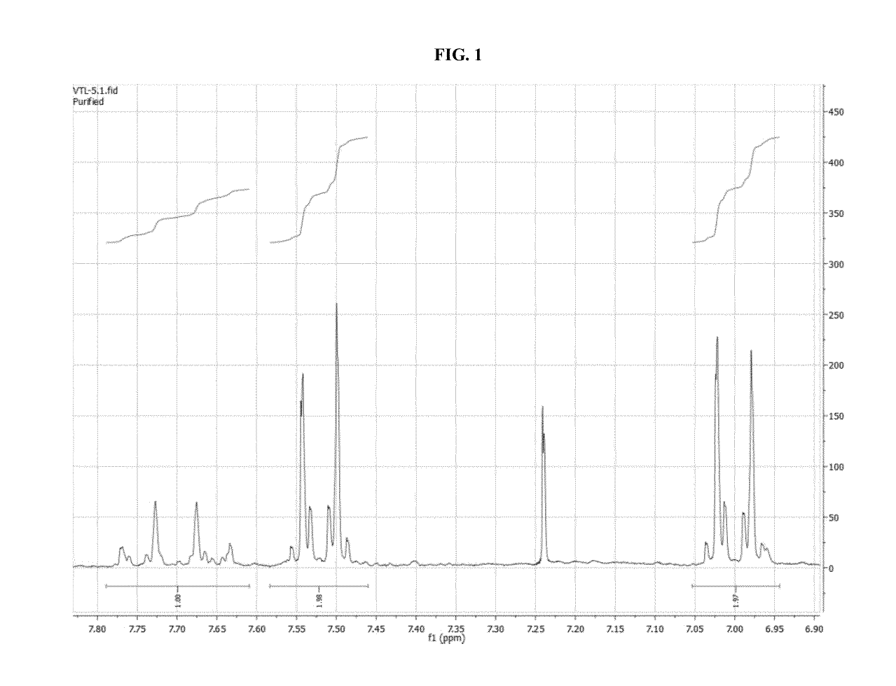

CuCl and t-BuOK (and 1,10-phenanthroline if indicated) were weighed into a 5 mL round bottomed flask equipped with a magnetic stirrer bar and the flask was sealed with a septum. The flask was purged with argon and three cycles of evacuation/filling were performed. DMF (2 mL) was added, and the reaction mixture was stirred for 45 minutes. Fluoroform (in excess) was passed through a silica cartridge and bubbled through the mixture using a syringe needle. The aryl iodide in DMF (1 mL) was then added via cannula, and the reaction mixture was stirred for the indicated period of time and temperature. The crude product was filtered through a silica plug, and analyzed by ¹⁹F NMR with (trifluoromethyl)benzene as an internal standard. After purification, a 1:1 mixture of the title compound and the tert butyl ester was obtained. See FIG. 1. 4-(trifluoromethyl)benzonitrile: 4-iodobenzonitrile (73 mg, 0.32 mmol) was added to CuCl (30 mg, 0.3 mmol) and t-BuOK (120 mg, 1.07 mmol) following the general procedure. Subsequently the reaction mixture was stirred at room temperature overnight. (20 h, 80° C.). 4-(trifluoromethyl)benzonitrile was isolated in 40% yield: 1H NMR (200 MHz, CDCl₃): δ 7.75 (d, 2H, J 8 Hz) 7.65 (d, 2H, J 8 Hz). 19F NMR (200 MHz, CDCl3): δ −63.5. Analytical data was in accordance with those published earlier (M. Huiban et al., Nature Chem. 2013 5, 941-944; L. Zhu et al., Org Lett. 2013, 15 (11), 2648-2651; M. Tredwell et al., Chem. Int. Ed. 2012, 51(46), 11426-11437; P. J. Riss et al., Org. Biomol. Chem., 2012, 10, 6980-6986; P. J. Riss et al., 2011, 47, 11873-11875; M. R. Kilbourn et al., Int. J. Rad. Appl. Instrum. A, 1990, 41, 823-828; O. Josse et al., Bioorg. Med. Chem. 2001, 9, 665-675; W. R. Dolbier Jr et al., Appl. Radiat. Isotopes 2001, 54:73-80; A. Lishchynskyi et al., J. Orga. Chem. 2013, 78, 11126-11146; O. A. Tomashenko, V. V. Grushin, Chem. Rev. 2011, 111, 4475; E. A. Symons et al., J Am Chem Soc 1981, 103, 3127-30).

4-(trifluoromethyl)benzamide: 4-iodobenzonitrile (85 mg, 0.34 mmol) was added to CuCl (35 mg, 0.35 mmol), t-BuOK (57 mg, 0.51 mmol) and 1,10-phenanthroline (75 mg, 0.42 mmol) following the general procedure (22 h, 60° C.). 19F NMR (200 MHz, unlocked): 6-63.5. Analytical data was in accordance with those published earlier 1,2 4-(trifluoromethyl)-1,1′-biphenyl: 4-iodobiphenyl (110 mg, 0.39 mmol) was added to CuCl (36 mg, 0.36 mmol), t-BuOK (53 mg, 0.47 mmol)) and 1,10-phenanthroline (66 mg, 0.37 mmol) following the general procedure (20 h, 60° C.). 19F NMR (200 MHz, unlocked): 6-61.3. Analytical data was in accordance with those published earlier (M. Huiban et al., Nature Chem. 2013 5, 941-944; L. Zhu et al., Org Lett. 2013, 15 (11), 2648-2651; M. Tredwell et al., Chem. Int. Ed. 2012, 51(46), 11426-11437; P. J. Riss et al., Org. Biomol. Chem., 2012, 10, 6980-6986; P. J. Riss et al., 2011, 47, 11873-11875; M. R. Kilbourn et al., Int. J. Rad. Appl. Instrum. A, 1990, 41, 823-828; O. Josse et al., Bioorg. Med. Chem. 2001, 9, 665-675; W. R. Dolbier Jr et al., Appl. Radiat. Isotopes 2001, 54:73-80; A. Lishchynskyi et al., J. Orga. Chem. 2013, 78, 11126-11146; O. A. Tomashenko, V. V. Grushin, Chem. Rev. 2011, 111, 4475; E. A. Symons et al., J Am Chem Soc 1981, 103, 3127-30).

Radiochemistry

Production of [¹⁸F]Fluoride Ion

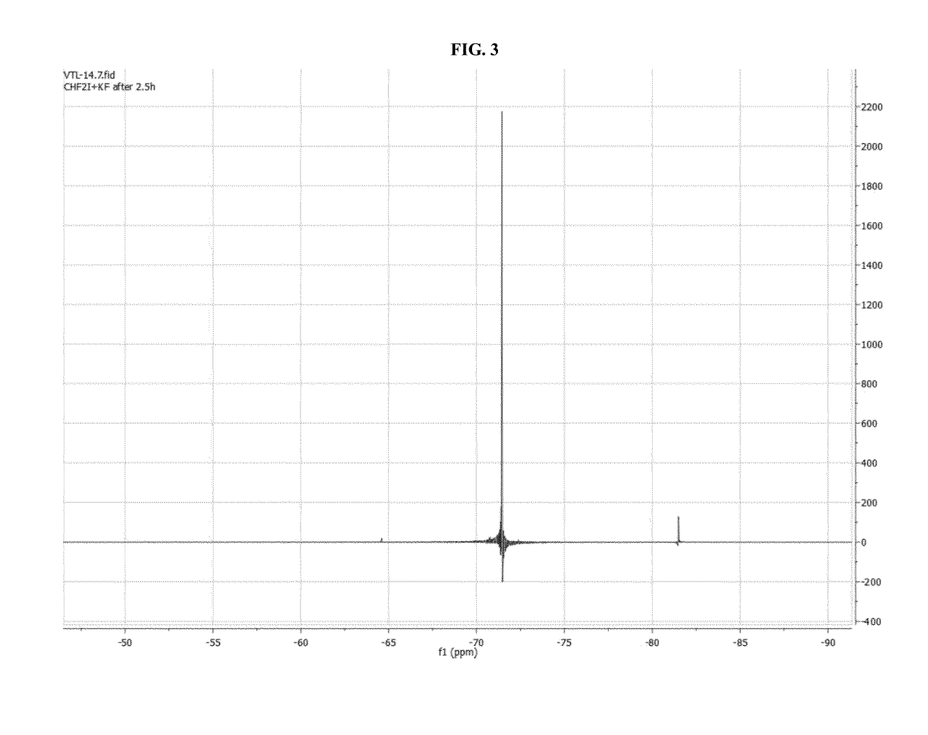

No-carrier-added fluorine-18 was produced on a PETtrace cyclotron (GE Healthcare, Uppsala, Sweden) using the 18O(p,n)¹⁸F reaction on an H₂ ¹⁸O liquid target (1.8 mL) with a proton beam (16.5; 3 MeV) for 5 min. At the end of the irradiation, the target water was diluted and passed through a Waters Accell plus light QMA solid phase extraction cartridge and the radioactive product was eluted at room temperature. Typically, [¹⁸F]fluoride ion (5.1 GBq, 1.8 mL) was diluted to 6 to 12 mL and subsequently trapped on Sep-Pak® Accell® Plus QMA Plus Light cartridges pre-conditioned with 1M potassium carbonate solution (5 mL), rinsed with water (10 mL) and purged with air. The [¹⁸F]fluoride ion was eluted to an individual vessel using a solution of Kryptofix K2.2.2 (35 μM) and an inorganic base, e.g. KHCO₃ (13 μM) in MeCN—H₂O, 9:1 (1 mL). The solutions were evaporated to dryness in a stream of Argon at 90° C. for 5 min. Residual water was removed by azeotropic co-evaporation using two portions of anhydrous MeCN (2×1 mL) for 5 min each See FIG. 3 for a setup for parallel evaporations.

TABLE 2
Retention factors from TLC, spotted in DMF and retention times from HPLC

| | TLC | HPLC |
|---|---|---|
| Radiotracer | R_{f} | tᵣ (min) |
| [18F]4-tert-butylbenzotrifluoride | 0.75 (n-hexane) | 1.8 (system C) |
| [18F]4-(trifluormethyl)benzonitrile | 0.96 (n-hexane-ethyl acetate, | 5.2 (system A) |
| | 1:1) | |
| [18F]Methyl 4-(trifluormethyl)benzoate | 0.80 (n-hexane-ethyl acetate, | 1.90 (system B) |
| | 8:2) | |
| [18F]4-Nitrobenzotrifluoride | 0.78 (n-hexane-ethyl acetate, | 7.6 (system A) |
| | 8:2) | |
| [18F]4-(trifluoromethyl)pyridine | 0.76 (n-hexane-ethylacetate, | 1.74 (system A) |
| | 6:4) | |
| [18F]Methyl 3-(trifluoromethyl)benzoate | 0.81 (n-hexane-ethyl acetate, | 1.83 (system B) |
| | 8:2) | |
| [18F]4-trifluoromethylbiphenyl | 0.7 (n-hexane) | 2.0 (system C) |
| [18F]4-(trifluoromethyl)benzamide | 0.75 (n-hexane-ethyl acetate, | 1.35 (system A) |
| | 1:1) | |
| [18F]4-benzyloxybenzotrifluoride | 0.75 (n-hexane-ethyl acetate, | 5.69 (system B) |
| | 8:2) | |
| [18F]4-(trifluormethyl)phenol | 0.51 (n-hexane-ethyl acetate, | 3.46 (system B) |
| | 8:2) | |
| [18F]3,5-Dimethylbenzotrifluoride | 0.73 (n-hexane) | 1.36 (system C) |
| [18F]2,5-Dimethylbenzotrifluoride | 0.75 (n-hexane) | 1.35 (system C) |
| [¹⁸F]1,3-dimethyl-5- | 0.88 (ethyl acetate) | 1.08 (system A) |
| (trifluoromethyl)pyrimidine-2,4-dione | | |

Measurements of Specific Radioactivities.

Specific radioactivities of representative, final radioactive products were determined by HPLC. A calibration curve was constructed for the HPLC UV absorbance signal versus concentration of non-radioactive reference. A sample of known radioactivity was then analyzed by HPLC. The area of the UV absorbance peak was converted into mass of the carrier. Specific activity (Ci/μmol; MBq/nmol) was then computed as the ratio of radioactivity in the injected sample to the mass of injected substance (μmol), corrected for physical decay to the end of synthesis. Deviations between radioHPLC and radioTLC result from byproducts, absorbtion of material on the HPLC column and/or volatility of some organic compounds.

Preparation and Drying of [¹⁸F]Fluoride Ion from Target Water; Screening Reactions

[¹⁸F]fluoride ion in water was produced by the ¹⁸O (p,n)¹⁸F nuclear reaction using a GE PETtrace cyclotron. After irradiation, [¹⁸F]fluoride ion (MBq range, 1 mL dilution in water) was trapped on a Sep-Pak® Accell® Plus QMA Plus Light Cartridge, which had been conditioned with 1M potassium carbonate solution (5 mL), rinsed with water (10 mL) and rinsed with air (30 mL). The [¹⁸F]fluoride ion was eluted into a standard V-bottom reaction vessel using a solution of Kryptofix K2.2.2 (35 μM) and KHCO₃ (13 μM) in MeCN—H₂O, 9:1 (1 mL). Up to six of these solutions were evaporated to dryness in a stream of Argon at 90° C. for 5 min. Residual water was removed by azeotropic co-evaporation using two portions of anhydrous MeCN (2×1 mL) for 5 min each. See FIG. 3 for a photographic image of the set up.

General Procedure for Screening Reactions:

CuX (58 μmol) was added to the dried [¹⁸F]fluoride ion complex and the vial was flushed with argon for 2 min. An ice cooled solution of DIPEA (10 μl, 59 μmol), iodobenzene (42 μmol) and difluoro(iodo)methane (20 μl, 169 μmol) in dry DMF (300 μl) were added via syringe. The sealed vial was heated at 145° C. for 10 min. The sample was analyzed by radio HPLC (Chromolith Performance (RP18e 100-4,6 mm), acetonitrile/water=7/3, flow rate 3 ml/min, 254 nm) and radioTLC (Raytest mini Gita, Gina Star TLC, Silica gel 60, solvent as indicated in Table 2) after cooling in a −25° C. freezer.

### [¹⁸F]-(4-methoxyphenyl)-[2-methyl-1-(2-morpholinoethyl)-6-(trifluoromethyl)indol-3-yl]methanone

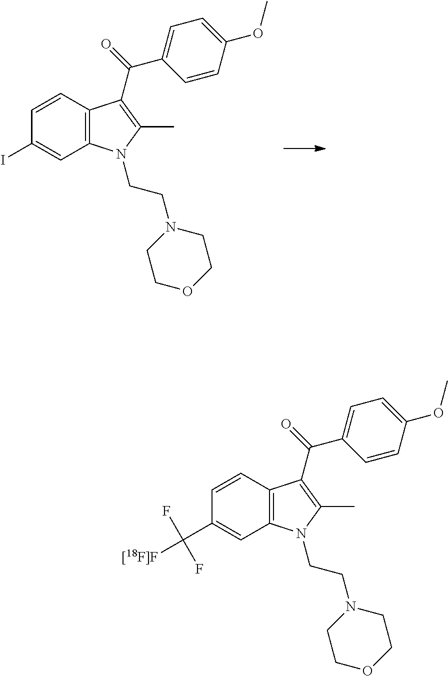 [0103] 1. [¹⁸F]KF (981 MBq)/kryptofix 2.2.2 (35 μmol), potassium hydrogen carbonate (13 μmol)
   CuBr (58 μmol)
   Ar, ˜2 min

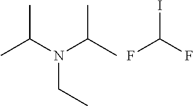

2. DIPEA (59 μmol)
   aryl iodide (21 μmol)
   CHF₂I (20 μl˜169 μmol)
   dry DMF (300 μl), Ar, 145° C., 10 min

Procedure

CuBr (8 mg, 58 μmol) was added to [¹⁸F]KF and the vial was flushed with argon for 2 min. An ice cooled solution of DIPEA (10 μl, 59 μmol), [6-iodo-2-methyl-1-(2-morpholinoethyl)indol-3-yl]-(4-methoxyphenyl)methanone (11 mg, 21 μmol) and difluoro(iodo)methane (20 μl in 169 μmol) in dry DMF (300 μl) were added via syringe. The sealed vial was heated at 145° C. for 10 min. A sample was withdrawn from the reaction mixture and analysed by radio HPLC and radio TLC (n-hexane-ethyl acetate; 1:1) after cooling in a −25° C. freezer. The reaction mixture was purified and the product was isolated using a Waters Sep-Pak Plus Silica Cartridge (n-hexane-ethyl acetate; 1:1).

### [¹⁸F]1-[5-(trifluoromethyl)-2-pyridyl]piperazine

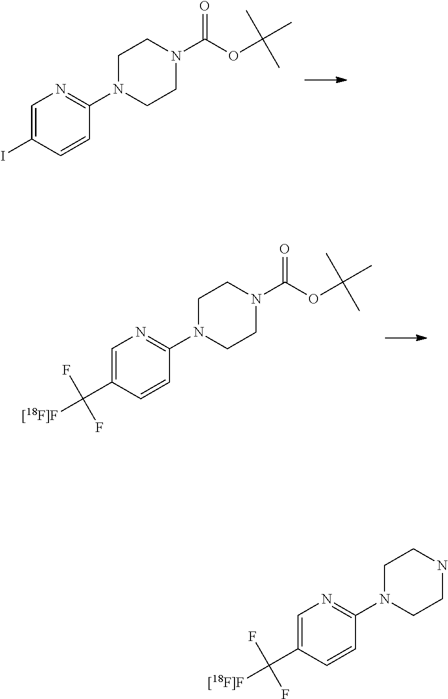 [0112] 1. [¹⁸F]KF (865 MBq)/kryptofix 2.2.2 (35 μmol), potassium hydrogen carbonate (13 μmol)
   CuBr (58 μmol)
   Ar, ˜2 min

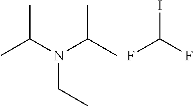

2. DIPEA (59 μmol)
   aryl iodide (38 μmol)
   CHF₂I (20 μl˜169 μmol)
   dry DMF (300 μl), Ar, 145° C., 10 min
3. TFA (100 μl, 1298 μmol), 90° C., 2 min
   dry ACN, 90° C., 5 min

Procedure

CuBr (8 mg, 58 μmol) was added to [¹⁸F]KF and the vial was flushed with argon for 2 min. An ice cooled solution of DIPEA (10 μl, 59 μmol), tert-butyl 4-(5-iodo-2-pyridyl)piperazine-1-carboxylate (15 mg, 38 μmol) and difluoro(iodo)methane (20 μl˜169 μmol) in dry DMF (300 μl) were added via syringe. The sealed vial was heated at 145° C. for 10 min. The sample was analyzed by radio HPLC and radio TLC (n-hexane/ethyl acetate=1/1) after cooling in a −25° C. freezer. The reaction mixture was purified, the sample was isolated using Waters Sep-Pak Plus Silica Cartridge (fractional elution with n-hexane-ethyl acetate; 1:1). The solvent was removed in a stream of Argon at 90° C. for 5 min and trifluoroacetic acid (100 μl) was added to the crude residue of [¹⁸F]-tert-butyl 4-[5-(trifluoromethyl)-2-pyridyl]piperazine-1-carboxylate and heated at 90° C. for 2 min. Dry acetonitrile (1000 μl) was added and the solvent was evaporated to dryness in a stream of Argon at 90° C. for 5 min. The sample was analyzed by radio HPLC and radio TLC (n-hexane-ethyl acetate; 1:1) after dilution with acetonitrile (150 μl) using a Discovery® HS F5 HPLC Column (250×4.6 mm, acetonitrile-water; 1:1), flow rate 1.5 ml/min.

### [¹⁸F]2-(trifluoromethyl)pyridine

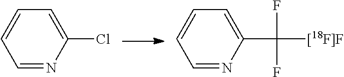 [0124] 1. [¹⁸F]KF (75 MBq)/kryptofix 2.2.2 (35 μmol), potassium hydrogen carbonate (13 μmol)
   CuBr (58 μmol)
   Ar, ˜2 min

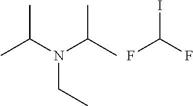

2. DIPEA (59 μmol)
   heteroaryl chloride (64 μmol)
   CHF₂I (20 μl˜169 μmol)
   dry DMF (300 μl), Ar, 145° C., 10 min

Procedure

CuBr (8 mg, 58 μmol) was added to [¹⁸F]KF and the vial was flushed with argon for 2 min. An ice cooled solution of DIPEA (10 μl, 59 μmol), 2-chloropyridine (7 mg, 64 μmol) and difluoro(iodo)methane (20 μl˜169 μmol) in dry DMF (300 μl) were added via syringe. The sealed vial was heated at 145° C. for 10 min.

The sample was measured by radio HPLC and radio TLC (ethyl acetate) after cooling in a −25° C. freezer.

### [¹⁸F]1,3-dimethyl-5-(trifluoromethyl)pyrimidine-2,4-dione

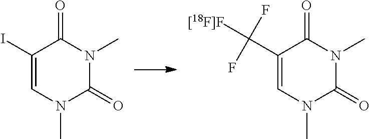 [0134] 1. [¹⁸F]KF (79 MBq)/kryptofix 2.2.2 (35 μmol), potassium hydrogen carbonate (13 μmol)
   CuBr (58 μmol)
   Ar, ˜2 min

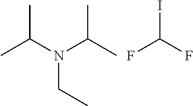

2. DIPEA (59 μmol)
   aryl iodide (38 μmol)
   CHF₂I (20 μl˜169 μmol)
   dry DMF (300 μl), Ar, 145° C., 10 min

### Procedure

CuBr (8 mg, 58 μmol) was added to [¹⁸F]KF and the vial was flushed with argon for 2 min. An ice cooled solution of DIPEA (10 μl, 59 μmol), 5-iodo-1,3-dimethyl-pyrimidine-2,4-dione (10 mg, 38 μmol) and difluoro(iodo)methane (20 μl˜169 μmol) in dry DMF (300 μl) were added via syringe. The sealed vial was heated at 145° C. for 10 min.
The sample was measured by radio HPLC and radio TLC (ethyl acetate) after cooling in a −25° C. freezer.

The key aim of the effort was to provide an operationally efficient one-reactor method for ¹⁸F-trifluoromethylation. Following methodological optimization a new Cu-mediated methodology for C[¹⁸F]CF3 bond formation directly involving [¹⁸F]fluoride ion in one pot with difluoro(iodo)methane was developed.

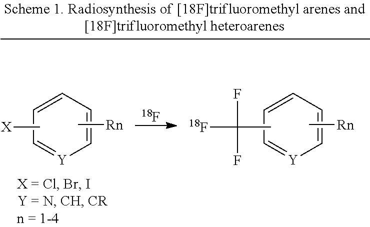

1. Synthesis of [¹⁸]trifluoromethane.

As a first step, the low boiling starting material CHF₂I (b.p. 22° C.) was substituted with a higher boiling difluoromethyl sulfonate in order to permit better control of the reaction stoichiometry and ease handling of the reagent. However, neither difluoromethyl tosylate nor difluoromethyl triflate were found to react to the desired product under a variety of conditions. CHF₂I was thus selected for all further experiments.

2. Optimization of Complex Ligand.

In non-radioactive chemistry, trifluoromethylation reactions are generally carried out with ‘stoichiometric’ amounts of reagents, however, in the case of no-carrier added ¹⁸F-radiochemistry, only a trace amount (<1 μmol) of fluoride ion is present at any time, rendering bi-atomic reaction intermediates highly unrealistic. Considering the fact that CuF is only stable as a complex in solution and otherwise disproportionates to CuO and CuF² this may come as an advantage; since the development of a one-pot method would require both species, n.c.a. [¹⁸F]fluoride ion and Cu⁺ to coexist. Consequently, the most efficient Cu-ligand system in combination with the most frequently used source of ¹⁸Ffluoride, a combination of 4,7,13,16,21,24-Hexaoxa-1,10-diazabicyclo[8.8.8]hexacosan (crypt-222, K2.2.2), K₂CO₃, and ¹⁸F— was used as a starting point for investigations.

Earlier studies have used moderately basic pyridines, such as phenanthroline, as a ligand to form the active trifluoromethylation reagent in the presence of a strong base such as potassium tert.-butoxide to deprotonate CHF₃ (pka=27). The use of the latter, however, is not required in the presence of crypt-222/K₂CO₃ which in itself is strong enough to deprotonate the trace amounts of [18F]CF₃H. Grushin and coworkers described that excess KOtBu ((CH₃)₃COK) would permit omission of a ligand under stoichiometric conditions. Unfortunately, their findings did not translate well into n.c.a radiochemistry (Table 7). A CuI-ligand system capable of mediating the trifluoromethylation reaction without affecting the in situ formation of trifluoromethane was investigated. As a model reaction, a 2.8:0.6:1:1 molar ratio of CHF₂I, 4-iodobenzonitrile CuI, ligand (see table 7) was used in order to establish working conditions for 10 min at 145° C. in DMF (0.3 mL). In preliminary experiments, it was found that a temperature of 145° C. was necessary to achieve rapid conversion. In control experiments omitting either ligand, or CHF₂I no ¹⁸F-labelled product was obtained, likewise, the use of triphenylphosphine (TPP) did result in traces (table 6). Surprisingly, neither pyridine derivative screened gave negligible yields of [18F]4-(trifluoromethyl)benzonitrile (Table 7). When the commercially available Cu— NHC ligand Bis(1,3-bis(2,6-diisopropylphenyl)imidazol-2-ylidene)copper(I) tetrafluoroborate (IPr.CuBF₄) was used, traces amounts of [18F]4-(trifluoromethyl)benzonitrile and an unknown side product were formed (Table 7). At this point it was determined that a slightly more basic ligand would be useful in dipolar aprotic media and aliphatic, tertiary amines were utilized.

This hypothesis was rewarded with the first double-figured yield when tetramethylethylenediamine (TMEDA), a ligand that had proved its value previously, was used ((M. Huiban et al., Nature Chem. 2013 5, 941-944; A. Lishchynskyi et al., J. Orga. Chem. 2013, 78, 11126-11146; O. A. Tomashenko, V. V. Grushin, Chem. Rev. 2011, 111, 4475; E. A. Symons et al., J Am Chem Soc 1981, 103, 3127-30).

Under these conditions (Table 3-7) [18F]4-(trifluoromethyl)benzonitrile was obtained in 19-48% radiochemical yield. Triethylamine (8%-28%, Table 3-4) which turned out to be inferior to TMEDA, was investigated. Further improved, albeit not yet satisfactory yield (3-47%, Table 1a-c) was achieved through the use of DBU. Further screening of ligand-catalyst combinations (Table 3-5) revealed N,N-diisopropylethylamine (DIPEA) to be very effective with regard to the formation of [18F]4-(trifluoromethyl)benzonitrile; without further optimization a radiochemical yield of 42-90% was achieved. No further ligand screening was conducted. The CuI-DIPEA system was used for future work.

### Ligand Screening

TABLE 3
Influence of ligand and basicity

| | TMEDA/ | | DBU/ | | DIPEA/ | |
|---|---|---|---|---|---|---|
| | Cs₂CO/K2.2.2/ | | Cs₂CO₃/K2.2.2/ | | Cs₂CO₃/K2.2.2/ | |
| | CuI | | CuI | | CuI | |
| | HPLC | TLC | HPLC | TLC | HPLC | TLC |
| Radiotracer | [%] | [%] | [%] | [%] | [%] | [%] |
| 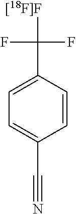 | 34 | 48 | 19 | 27 | 52 | 83 |

TABLE 4
Influence of ligand and basicity

| | TMEDA/ | | DBU/ | | DIPEA/ | | NeT₃/ | |
|---|---|---|---|---|---|---|---|---|
| | KHCO₃/K2.2.2/CuI | | KHCO₃/K2.2.2/CuI | | KHCO₃/K2.2.2/CuI | | KHCO₃/K2.2.2/CuI | |
| | HPLC | TLC | HPLC | TLC | HPLC | TLC | HPLC | TLC |
| Radiotracer | [%] | [%] | [%] | [%] | [%] | [%] | [%] | [%] |
| 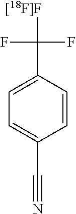 | 26 | 35 | 32 | 47 | 52 | 90 | 7 | 8 |

TABLE 5
Influence of ligand and basicity

| | TMEDA/ | | DBU/ | | DIPEA/ | | NEt₃ | |
|---|---|---|---|---|---|---|---|---|
| | K₂CO₃/K2.2.2/CuI | | K₂CO₃/K2.2.2/CuI | | K₂CO₃/K2.2.2/CuI | | K₂CO₃/K2.2.2/CuI | |
| | HPLC | TLC | HPLC | TLC | HPLC | TLC | HPLC | TLC |
| Radiotracer | [%] | [%] | [%] | [%] | [%] | [%] | [%] | [%] |
| 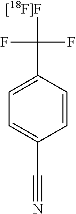 | 23 | 19 | 3 | 3 | 28 | 42 | 26 | 28 |

TABLE 6
Influence of ligand and basicity

| | Pyridine/ | | DMAP/ | | Phenanthroline/ | | Bipyridine/ | |
|---|---|---|---|---|---|---|---|---|
| | K₂CO₃/K2.2.2/CuI | | K₂CO₃/K2.2.2/CuI | | K₂CO₃/K2.2.2/CuI | | K₂CO₃/K2.2.2/CuI | |
| | HPLC | TLC | HPLC | TLC | HPLC | TLC | HPLC | TLC |
| Radiotracer | [%] | [%] | [%] | [%] | [%] | [%] | [%] | [%] |
| 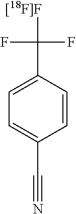 | 1 | 2 | 9 | 9 | 6 | 5 | 10 | 8 |

TABLE 7
Influence of ligand and basicity

| | TPP | | (CH₃)₃COK/ | | IPr•CuBF₄/ | |
|---|---|---|---|---|---|---|
| | K₂CO₃/ | | K₂CO₃/ | | DIPEA | |
| | K2.2.2/CuI | | K2.2.2/CuI | | K₂CO₃/K2.2.2/CuI | |
| | HPLC | TLC | HPLC | TLC | HPLC | TLC |
| Radiotracer | [%] | [%] | [%] | [%] | [%] | [%] |
| 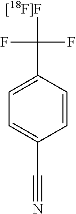 | 1 | 1 | 3 | 2 | 2 | (21) |

3. Effect of Fluoride Ion Source.

Under the screened conditions, DIPEA was found to be generally superior to TMEDA and DBU. Substitution of the cryptand crypt-222 (Table 9) by the corresponding crown ether 18-crown-6 (Table 10) led to a slightly improved radiochemical yield from 42% of about 49%. Whereas the use of tetrabutylammonium hydroxide (TBAOH) to form tetrabutylammonium fluoride (TBA[18F]F) (Table 9) did not have any benefit (2%)., tetraethylammonium carbonate (TEA₂CO₃) to essentially obtain tetraethylammonium fluoride (TEA[18F]F) had a remarkable impact (56%) (Table 10) However, the use of Cs₂CO₃, crypt-222 and DIPEA as base, led to a remarkable increase in radiochemical yields in the formation of [18F]4-(trifluoromethyl)benzonitrile (83%, Table 9). In the end these conditions were second only to the combination of KHCO₃, crypt-222 and DIPEA which resulted in more than 76% (Table 9). Cs₂CO₃, crypt-222 and KHCO₃, crypt-222 were established as the preferred fluoride ion source.

### Base Screening

TABLE 8
Influence of basicity

| | K₂CO₃/ | | KHCO₃/ | | Cs₂CO₃/ | | | |
|---|---|---|---|---|---|---|---|---|
| | K2.2.2/ | | K2.2.2/ | | K2.2.2/ | | Bu₄NOH/ | |
| | TMEDA/ | | TMEDA/ | | TMEDA/ | | TMEDA/ | |
| | CuI | | CuI | | CuI | | CuI | |
| | HPLC | TLC | HPLC | TLC | HPLC | TLC | HPLC | TLC |
| Radiotracer | [%] | [%] | [%] | [%] | [%] | [%] | [%] | [%] |
| 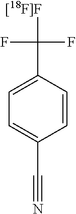 | 23 | 19 | 27 | 48 | 33 | 38 | 15 | 18 |

TABLE 9
Influence of basicity

| | K₂CO₃/ | | KHCO₃/ | | Cs₂CO₃/ | | Bu₄NOH/ | |
|---|---|---|---|---|---|---|---|---|
| | K2.2.2/ | | K2.2.2/ | | K2.2.2/ | | DIPEA/ | |
| | DIPEA/CuI | | DIPEA/CuI | | DIPEA/CuI | | CuI | |
| | HPLC | TLC | HPLC | TLC | HPLC | TLC | HPLC | TLC |
| Radiotracer | [%] | [%] | [%] | [%] | [%] | [%] | [%] | [%] |
| 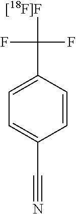 | 28 | 42 | 36 | 76 | 52 | 83 | 2 | 2 |

TABLE 10
Influence of basicity

| | K₂CO₃/18-Crown- | | | |
|---|---|---|---|---|
| | 6/ | | (CH₃CH₂)₄N(HCO₃) | |
| | DIPEA/CuI | | DIPEA/CuI | |
| | HPLC | TLC | HPLC | TLC |
| Radiotracer | [%] | [%] | [%] | [%] |
| 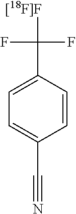 | 42 | 49 | 49 | 56 |

3. Reaction Time and Solvent.

In order to further optimize the reaction outcome, the contribution of the reaction time was investigated. Increasing the reaction time beyond 10 minutes did not improve the yield (Table 12). Substitution of DMF with DMSO or THF were detrimental (Table 11) both of these solvents were ineffective. However, substitution of DMF for MeCN provided a viable alternative and similar yields were obtained. However, the main culprit of using MeCN under these conditions is the fairly pronounced pressure build up in the reactor, which may results in difficulties during automation. In the case of acetonitrile an increased loss of activity was observed.

In essence, aryl iodides were confirmed to be the most appropriate halides for this synthesis (Table 12), a steep decline in radiochemical yield occurred when switching from 4-iodobenzonitrile (56%, Table 12) to the corresponding 4-bromobenzonitrile (1% Table 4) and 4-chlorobenzonitrile (1%, Table 12).

### Solvent Screening

TABLE 11
Influence of solvent

| | ACN/ | | THF/ | | DMF/ | | DMSO/ | |
|---|---|---|---|---|---|---|---|---|
| | CuI/KHCO₃/K2.2.2/ | | CuI/KHCO₃/K2.2.2/ | | CuI/KHCO₃/K2.2.2/ | | CuI/KHCO₃/K2.2.2/ | |
| | DIPEA | | DIPEA | | DIPEA | | DIPEA | |
| | HPLC | TLC | HPLC | TLC | HPLC | TLC | HPLC | TLC |
| Radiotracer | [%] | [%] | [%] | [%] | [%] | [%] | [%] | [%] |
| 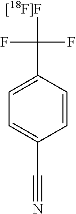 | 16 | 78 | 0 | 8 | 36 | 76 | 0 | 0 |

### Time Screening

TABLE 12
Influence of time

| | 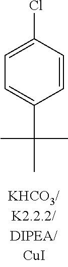 | | 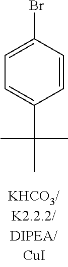 | | 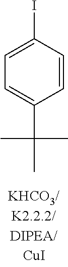 | | 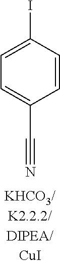 | |
|---|---|---|---|---|---|---|---|---|
| | HPLC | TLC | HPLC | TLC | HPLC | TLC | HPLC | TLC |
| Time | [%] | [%] | [%] | [%] | [%] | [%] | [%] | [%] |
| 10 min | 0 | 1 | 0 | 1 | 14 | 56 | 31 | 83 |
| 20 min | 0 | 0 | 0 | 1 | 16 | 53 | 26 | 82 |

5. Variation of Copper Catalyst Source.

It was tested whether CuI was the preferred source of copper catalyst by changing the copper salt in the promising reaction example that used DIPEA-CuI (Table 13). Reaction did not occur when CuI was omitted. Equimolar replacement of CuI with CuCl, CuOAc, CuCN, or fluorotristriphenylphosphine CuI led to dimished radiochemical yield (Table 13-14). Also arene complexes of CuOTf (Table 13-14) were not effective in the absence of DIPEA or gave only trace ¹⁸F labelled product (Table 14). CuBr led to excellent radiochemical yield with 89%, close to Tetrakis acetonitrile CuOTf, which provided the highest yield with 93% (Table 15). CuBr and Tetrakis acetonitrile CuOTf were established as the preferred copper source.

### Copper Salt Screening

TABLE 13
Influence of copper(I) source

| | CuCl/ | | CuBr/ | | CuI/ | | CuCN/ | |
|---|---|---|---|---|---|---|---|---|
| | KHCO₃/K2.2.2/DIPEA | | KHCO₃/K2.2.2/DIPEA | | KHCO₃/K2.2.2/DIPEA | | KHCO₃/K2.2.2/DIPEA | |
| | HPLC | TLC | HPLC | TLC | HPLC | TLC | HPLC | TLC |
| Radiotracer | [%] | [%] | [%] | [%] | [%] | [%] | [%] | [%] |
| 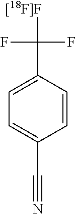 | 4 | 40 | 51 | 89 | 36 | 76 | 7 | 60 |

TABLE 14
Influence of copper(I) source

| | | | | | Fluorotris- | | | |
|---|---|---|---|---|---|---|---|---|
| | | | | | (triphenylphosphine) | | | |
| | Cuac/ | | Cu(CH₃CN)₄xCF₃SO₃/ | | copper(I)/ | | (CF₃SO₃Cu)₂xC₆H₆/ | |
| | KHCO₃/K2.2.2/DIPEA | | KHCO₃/K2.2.2 | | KHCO₃/K2.2.2 | | KHCO₃/K2.2.2 | |
| | HPLC | TLC | HPLC | TLC | HPLC | TLC | HPLC | TLC |
| Radiotracer | [%] | [%] | [%] | [%] | [%] | [%] | [%] | [%] |
| 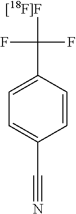 | 7 | 10 | 4 | 5 | 0 | 0 | 0 | 0 |

TABLE 15
Influence of copper(I) source

| | (CF₃SO₃Cu)₂xC₆H₅CH₃/ | | Cu(CH₃CN)₄xCF₃SO₃KHCO₃/ | |
|---|---|---|---|---|
| | KHCO₃/K2.2.2 | | K2.2.2/DIPEA | |
| | HPLC | TLC | HPLC | TLC |
| Radiotracer | [%] | [%] | [%] | [%] |
| 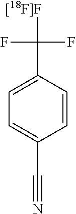 | 0 | 0 | 50 | 93 |

6. Investigation of Substrate Scope.

General conditions as optimised above were used to investigate the substrate scope of the ¹⁸F-trifluoromethylation. A variety of commercially available aryl iodides were screened (Table 16-18). Most assayed functional groups were found to be compatible with the reaction conditions. Potentially sensitive substrates such as 4-iodobenzonitrile or methyl 4-iodobenzoate, which might be sensible to exposure to carbanionic forms of trifluoromethylating reagents, gave the desired radioactive products in high to excellent yields. Even 4-iodophenol containing a protic hydroxyl group was tolerated to some extent. The protic 4-iodobenzamide gave low yield and two unidentified by-products were observed in the reaction mixture. Electron deficient substrates globally resulted in slightly higher radiochemical yields compared to electron-rich arenes.

### Substance Screening

TABLE 16
Influence of functional groups under different conditions

| | | | | | KHCO₃/K2.2.2/ | | | |
|---|---|---|---|---|---|---|---|---|
| | K₂CO₃/K2.2.2/TMEDA | | KHCO₃/K2.2.2/DIPEA | | DIPEA/ | | KHCO₃/K2.2.2/DIPEA/ | |
| | CuI | | CuI | | Cu(CH₃CN)₄xCF₃SO₃ | | CuBr | |
| | HPLC | TLC | HPLC | TLC | HPLC | TLC | HPLC | TLC |
| Radiotracer | [%] | [%] | [%] | [%] | [%] | [%] | [%] | [%] |
| 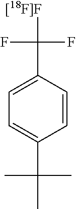 | 13 | 8 | 8 | 41 | 29 | 84 | 28 | 63 |
| 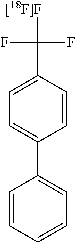 | 18 | 16 | 12 | 68 | 28 | 85 | 22 | 85 |
| 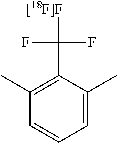 | na | na | 40 | 47 | 46 | 76 | 77 | 81 |
| 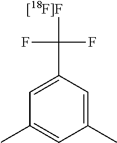 | na | na | 17 | 40 | 27 | 52 | 50 | 69 |

TABLE 17
Influence of functional groups under different conditions

| | K₂CO₃/K2.2.2/TMEDA/ | | KHCO₃/K2.2.2/DIPEA/ | | KHCO₃/K2.2.2/DIPEA/ | | KHCO₃/K2.2.2/DIPEA/ | |
|---|---|---|---|---|---|---|---|---|
| | CuI | | CuI | | Cu(CH₃CN)₄xCF₃SO₃ | | CuBr | |
| | HPLC | TLC | HPLC | TLC | HPLC | TLC | HPLC | TLC |
| Radiotracer | [%] | [%] | [%] | [%] | [%] | [%] | [%] | [%] |
| 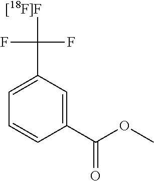 | na | na | 24 | 64 | 42 | 87 | 57 | 91 |
| 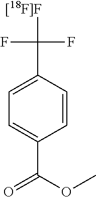 | na | na | 33 | 72 | 56 | 86 | 69 | 91 |
| 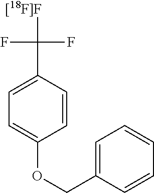 | na | na | na | 69 | 34 | 84 | 47 | 91 |
| 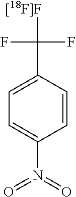 | na | na | 31 | 68 | 56 | 88 | 62 | 91 |
| 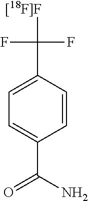 | 12 | 4 | 12 | 38 | 12 | 18 | 39 | 54 |

TABLE 18
Influence of functional groups under different conditions

| | K₂CO₃/K2.2.2/TMEDA/ | | KHCO₃/K2.2.2/DIPEA/ | | KHCO₃/K2.2.2/DIPEA/ | | KHCO₃/K2.2.2/DIPEA/ | |
|---|---|---|---|---|---|---|---|---|
| | CuI | | CuI | | Cu(CH₃CN)₄xCF₃SO₃ | | CuBr | |
| | HPLC | TLC | HPLC | TLC | HPLC | TLC | HPLC | TLC |
| Radiotracer | [%] | [%] | [%] | [%] | [%] | [%] | [%] | [%] |
| 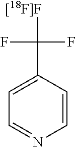 | 10 | 3 | 31 | 36 | 33 | 51 | 44 | 70 |
| 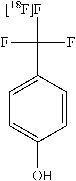 | na | na | 8 | 14 | 16 | 7 | 20 | 12 |
| 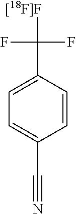 | 24 | 25 | 42 | 86 | 50 | 93 | 50 | 94 |

7. Translation of the Method: Labelling of Propective Radiotracers.

Having confirmed that it was possible to prepare a variety of [¹⁸F]trifluoromethyl arenes efficiently within only 10 min from the end of radionuclide production, the feasibility of synthesizing prospective radiotracer candidates bearing molecular structures common for small molecule drugs was investigated (Table 19).

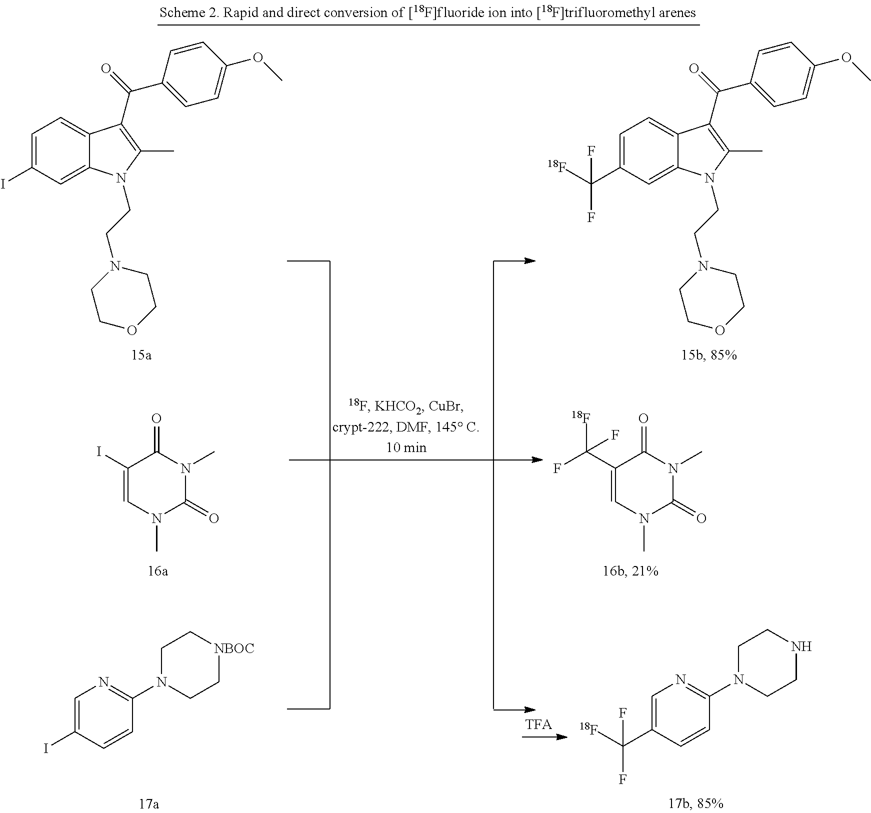

### Biomolecules

TABLE 19
Labelling of molecules with prospective biological activity

| | | KHCO₃/K2.2.2/DIPEA/ | |
|---|---|---|---|
| | | CuBr | |
| Precursor | Radiotracer | HPLC [%] | TLC [%] |
| 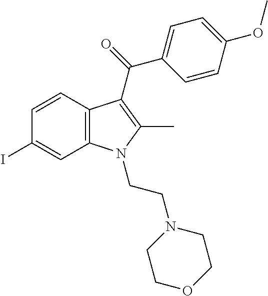 [SMILES: COc1ccc(C(=O)c2c(C)n(CCN3CCOCC3)c3cc(I)ccc23)cc1] | 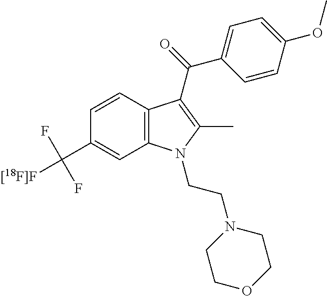 | 41 | 85 |
| 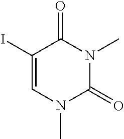 | 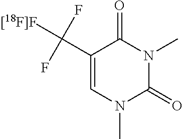 | 67 | 73 |
| 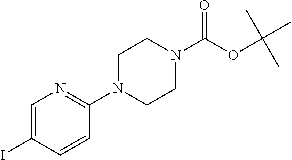 [SMILES: CC(C)(C)OC(=O)N1CCN(c2ccc(I)cn2)CC1] | 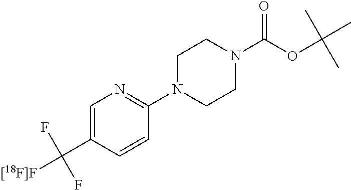 | 46 | 85 |

Treatment of the precursor [6-iodo-2-methyl-1-(2-morpholinoethyl)indol-3-yl]-(4-methoxyphenyl)methanone to synthesize the prospective subtype selective cannabinoid receptor agonist [18F](4-methoxyphenyl)-[2-methyl-1-(2-morpholinoethyl)-6-(trifluoromethyl)indol-3-yl]methanone which is of interest in the context of PET imaging of neuro-inflammation associated with a variety of medical conditions with ¹⁸F under standard conditions afforded [18F](4-methoxyphenyl)-[2-methyl-1-(2-morpholino ethyl)-6-(trifluoromethyl)indol-3-yl]methanone in 85% RCY. Likewise, the direct radiosynthesis of trifluorothymine derivate [¹⁸F]1,3-dimethyl-5-(trifluoromethyl)pyrimidine-2,4-dione from the corresponding iodide precursor 5-iodo-1,3-dimethyl-pyrimidine-2,4-dione was performed in order to provide this compound for ongoing cancer imaging efforts in rodent models of peripheral tumours. [¹⁸F]1,3-dimethyl-5-(trifluoromethyl)pyrimidine-2,4-dione was obtained in a radiochemical yield of 73%. In an extension of the concept the BOC-protected piperazine tert-butyl 4-(5-iodo-2-pyridyl)piperazine-1-carboxylate was converted into the BOC-protected piperazine[18F]tert-butyl 4-[5-(trifluoromethyl)-2-pyridyl]piperazine-1-carboxylate in 85% yield and in an further step deprotectet with TFA to the prospective 5-HT receptor radiotracer [¹⁸F]1-[5-(trifluoromethyl)-2-pyridyl]piperazine.

### ABBREVIATIONS

Cuac=Copper(I) actate
Cu(CH₃CN)₄xCF₃SO₃=Tetrakisacetonitrile copper(I) triflate
(CF₃SO₃Cu)₂xC₆H₆=Copper(I) trifluoromethanesulfonate benzene complex
(CF₃SO₃Cu)₂xC₆H₅CH₃=Copper(I) trifluoromethanesulfonate toluene complex
DBU=1,8-Diazabicyclo[5.4.0]undec-7-ene

### DMAP=4-(Dimethylamino)pyridine

### TMEDA=N,N,N′,N′-Tetramethylethylenediamine

NEt₃=triethylamine

### DIPEA=N,N-Diisopropylethylamine

(CH₃CH₂)₄N(HCO₃)=Tetraethylammonium bicarbonate

### K2.2.2=Kryptofix® 222

IPr.CuBF₄=Bis(1,3-bis(2,6-diisopropylphenyl)imidazol-2-ylidene)copper(I) tetrafluoroborate

### ACN=Acetonitrile

### DMF=N,N-Dimethylformamide

### THF=Tetrahydrofuran

DMSO=Dimethyl sulfoxide

### DMAP=4-(Dimethylamino)pyridine

### Phenanthroline=1,10-Phenanthroline

### Bipyridine=2,2′-Bipyridyl

### TPP=Triphenylphosphine

(CH₃)₃COK=Potassium tert-butoxide

This example describes CuI mediated ¹⁸F-trifluoromethylation reactions with difluoro(iodo)methane that are highly efficient in the presence of a simple combination of DIPEA, CuBr and iodoarene. This methodology was extended to three examples of a single-pot synthesis of candidate radioligands for PET imaging (Table 7). The resulting no carrier added [18F]trifluoromethyl arenes are obtained in sufficient yield in an operationally convenient protocol, suitable for straightforward automation. This direct and rapid conversion of iodoarenes is tolerant to diverse functional groups and consequently provides convenient access to a variety of drug molecules containing the CF₃-group. Given the high prevalence of the CF₃-group and its prominent role in drug development, paired with the availability of ¹⁸F at most PET centers, the methodology finds use in the development of PET radiotracers in particular from known, well characterized drug molecules.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A method of synthesizing a compound of the formula (I) or (II) or (III), 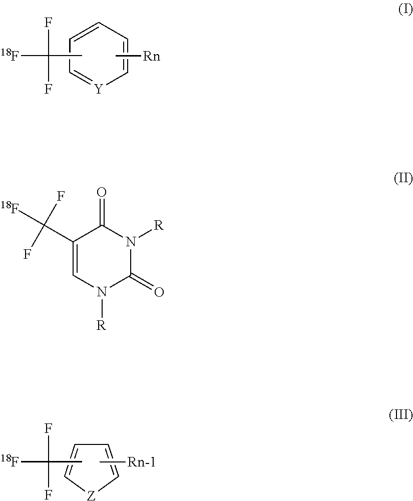
wherein Y═N, CH, or CR; wherein Z═NR, O, or S; and wherein R is one or more than one and dependent or independent of each other and selected from the group consisting of substituted, non-substituted, functionalized, non-functionalized H, halogen, nitro, nitril, isonitril, cyanate, isocyanate, hydroxyl, amide, alkyl, alkenyl, alkynyl, aryl, heteroaryl, alkyl-, aryl-, heteroaryl ether, alkyl-, aryl-, heteroaryl thioether, alkyl-, aryl-, heteroaryl ketone, alkyl-, aryl-, heteroaryl thioketone, alkyl-, aryl-, heteroaryl amide, alkyl-, aryl-, heteroaryl thioamide, alkyl-, aryl-, heteroaryl urea, alkyl-, aryl-, heteroaryl thiourea, alkyl-, aryl-, heteroaryl urethane, alkyl-, aryl-, heteroaryl thiourethane, alkyl-, aryl-, heteroaryl ester, alkyl-, aryl-, heteroaryl thioester, alkyl-, aryl-, heteroaryl amine, monocyclic, and multi cyclic, isotope containing and wherein n=1-4, comprising:
contacting base, ¹⁸F⁻ ion and a copper source, and a compound of the formula (IV) or (V) or (VI) wherein Y═N, CH, or CR and wherein Z═NR, O, or S and wherein X═Cl, Br, or I and wherein R is one or more than one and dependent or independent of each other and selected from substituted, non-substituted, functionalized, non-functionalized H, halogen, nitro, nitril, isonitril, cyanate, isocyanate, hydroxyl, amide, alkyl, alkenyl, alkynyl, aryl, heteroaryl, alkyl-, aryl-, heteroaryl ether, alkyl-, aryl-, heteroaryl thioether, alkyl-, aryl-, heteroaryl ketone, alkyl-, aryl-, heteroaryl thioketone, alkyl-, aryl-, heteroaryl amide, alkyl-, aryl-, heteroaryl thioamide, alkyl-, aryl-, heteroaryl urea, alkyl-, aryl-, heteroaryl thiourea, alkyl-, aryl-, heteroaryl urethane, alkyl-, aryl-, heteroaryl thiourethane, alkyl-, aryl-, heteroaryl ester, alkyl-, aryl-, heteroaryl thioester, alkyl-, aryl-, heteroaryl amine, monocyclic, and multi cyclic, isotope containing and wherein n=1-4, 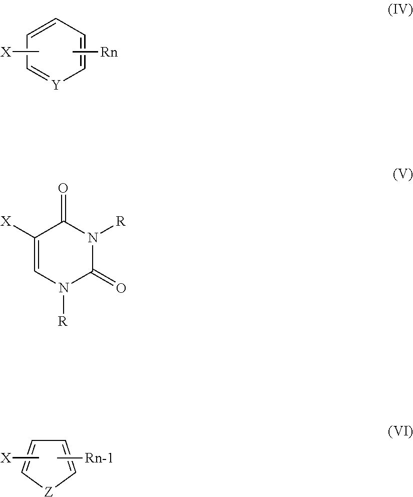
and a ligand and difluoro(iodo)methane, in a solvent a single reaction vessel; and incubating for an appropriate incubation time at an elevated temperature.

2. The method of claim 1, wherein said temperature is between 50° C. and 750° C.

3. The method of claim 2, wherein said reaction temperature is 145° C.

4. The method of claim 1, wherein said incubation time is between 1 s and 5 h.

5. The method of claim 4, wherein said incubation time is 10 minutes.

6. The method of claim 1, wherein said copper source is a copper(I) source.

7. The method of claim 6, wherein said copper source is selected from CuBr, Tetrakisacetonitrile copper(I) triflate and CuI.

8. The method of claim 1, wherein said solvent is a polar aprotic solvent.

9. The method of claim 8, wherein said polar aprotic solvent is selected from DMF, acetonitrile, and dialkyl ketone.

10. The method of claim 1, wherein said base is a metal carbonate and/or metal bicarbonate and cyptand.

11. The method of claim 10, wherein said base is selected from KHCO₃ and crypt-222, Cs₂CO₃ and crypt-222, K₂CO₃ and crypt-222, K₂CO₃ and 18-Crown-6, a nonmetal carbonate, a nonmetal bicarbonate, and tetraethylammonium bicarbonate

12. The method of claim 1, wherein said ligand is an organic non- to low-nucleophilic amine or phosphazene.

13. The method of claim 12, wherein said ligand is selected from DBU, TMEDA, NEt₃ and DIPEA.

14. The method of claim 12, wherein said ligand stabilized the copper mediate.

15. The method of claim 12, wherein said ligand is a base.

16. The method of claim 1, wherein said, when ¹⁹F-fluoride ion is present, the CF₃ substituted compounds are also synthesized.

17. A compound synthesized by the method of claim 1.

18. A method of PET imagining, comprising:
a) administering a compound of claim 1 to a subject, and
b) obtaining a PET image of said compound in said subject.
